(19) United States
(12) Patent Application Publication
(10) Pub. No.: US 2007/0244130 A1
(43) Pub. Date: Oct. 18, 2007

(54) Compounds and Compositions as Ppar Modulators

(75) Inventors: Robert Epple, San Diego, CA (US); Yongping Xie, San Diego, CA (US); Xing Wang, San Diego, CA (US); Christopher Cow, San Diego, CA (US); Ross Russo, Encinitas, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 11/597,260

(22) PCT Filed: May 24, 2005
(86) PCT No.: PCT/US05/18166

§ 371 Date: Jul. 5, 2007

Related U.S. Application Data
(60) Provisional application No. 60/574,137, filed on May 24, 2004.
(60) Provisional application No. 60/649,671, filed on Feb. 2, 2005.

(51) Int. Cl. A01N 43/54; A61K 31/42; A61K 31/435; C07D 263/30; C07D 401/00; C07D 411/00

(52) U.S. Cl. 514/256; 514/340; 514/374; 544/333; 546/269.4; 548/235

(57) ABSTRACT

The invention provides compounds, pharmaceutical compositions comprising such compounds and methods of using such compounds to treat or prevent diseases or disorders associated with the activity of the Peroxisome Proliferator-Activated Receptor (PPAR) families, particularly the activity of PPAR.

### CROSS-REFERENCE TO RELATED APPLICATIONS

This application claims the benefit of priority to U.S. Provisional Patent Application No. 60/574,137, filed 24 May 2004, and U.S. Provisional Patent Application No. 60/649,671, filed 2 Feb. 2005. The fall disclosures of these applications are incorporated herein by reference in their entirety and for all purposes.

### BACKGROUND OF THE INVENTION

1. Field of the Invention

The invention provides compounds, pharmaceutical compositions comprising such compounds and methods of using such compounds to treat or prevent diseases or disorders associated with the activity of the Peroxisome Proliferator-Activated Receptor (PPAR) families, particularly the activity of PPARδ.

2. Background

Peroxisome Proliferator Activated Receptors (PPARs) are members of the nuclear hormone receptor super family, which are ligand-activated transcription factors regulating gene expression. Certain PPARs are associated with a number of disease states including dyslipidemia, hyperlipidemia, hypercholesteremia, atherosclerosis, atherogenesis, hypertriglyceridemia, heart failure, myocardial infarction, vascular diseases, cardiovascular diseases, hypertension, obesity, inflammation, arthritis, cancer, Alzheimer's disease, skin disorders, respiratory diseases, ophthalmic disorders, IBDs (irritable bowel disease), ulcerative colitis and Crolm's disease. Accordingly, molecules that modulate the activity of PPARs, particularly PPARδ, are useful as therapeutic agents in the treatment of such diseases.

### SUMMARY OF THE INVENTION

In one aspect, the present invention provides compounds of Formula I:

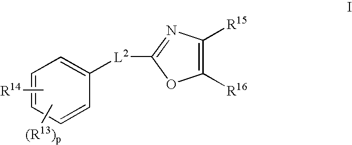

in which:

p is an integer selected from 0 to 3;

L² is selected from —XOX—, —XS(O)₀₋₂X— and —XS(O)₀₋₂XO—; wherein

X is independently selected from a bond and C₁₋₄alkylene; wherein any alkylene of L² can be optionally substituted by 1 to 3 radicals selected from halo, C₁₋₆alkyl, C₁₋₆alkoxy, halo-substituted-C₁₋₆alkyl and halo-substituted-C₁₋₆alkoxy;

R¹³ is selected from halo, C₁₋₆alkyl, C₁₋₆alkoxy, hydroxy-C₁₋₆alkyl, halo-substituted-C₁₋₆alkyl, halo-substituted-C₁₋₆alkoxy, C₆₋₁₀aryl, C₅₋₁₀heteraryl, C₃₋₁₂cycloalkyl and C₃₋₈heterocycloalkyl; wherein any aryl, heteroaryl, cycloalkyl and heterocycloalkyl of R¹³ is optionally substituted with 1 to 3 radicals independently selected from halo, nitro, cyano, C₁₋₆alkyl, C₁₋₆alkoxy, hydroxy-C₁₋₆alkyl, halo-substituted-C₁₋₆alkyl and halo-substituted-C₁₋₆alkoxy;

R¹⁴ is selected from —XOXC(O)OR¹⁷ and —XC(O)OR¹⁷; wherein X is a bond or C₁₋₄alkylene; and R¹⁷ is selected from hydrogen and C₁₋₆alkyl;

R¹⁵ and R¹⁶ are independently selected from —R¹⁸ and —YR⁸; wherein Y is a selected from C₁₋₆alkylene, C₂₋₆alkenylene, C₂₋₆alkynylene, —C(O)NR¹⁷— and —OX—; X is a bond or C₁₋₄alkylene; R¹⁷ is selected from hydrogen and C₁₋₆alkyl; and R¹⁸ is selected from C₃₋₁₂cycloalkyl, C₃₋₈heterocycloalkyl, C₆₋₁₀aryl and C₅₋₁₃heteroaryl; or R¹⁵ and R¹⁶ together with the atoms to which R¹⁵ and R¹⁶ are attached form fused bicyclic or tricyclic C₅₋₁₄heteroaryl;

wherein any aryl, heteroaryl, cycloalkyl and heterocycloalkyl of R¹⁸, or the combination of R¹⁵ and R¹⁶, is optionally substituted with 1 to 3 radicals independently selected from halo, nitro, cyano, C₁₋₆alkyl, C₁₋₆alkoxy, C₁₋₆alkylthio, hydroxy-C₁₋₆alkyl, halo-substituted-C₁₋₆alkyl, halo-substituted-C₁₋₆alkoxy, C₃₋₁₂cycloalkyl, C₃₋₈heterocycloalkyl, C₆₋₁₀aryl, C₅₋₁₃heteroaryl, —XS(O)₀₋₂R¹⁷, —XS(O)₀₋₂XR¹⁹, —XNR¹⁷R¹⁷, —XNR¹⁷S(O)₀₋₂R¹⁷, —XNR¹⁷C(O)R¹⁷, —XC(O)NR¹⁷R¹⁷, —XNR¹⁷C(O)R¹⁹, —XC(O)NR¹⁷R¹⁹, —XC(O)R¹⁹, —XNR¹⁷XR¹⁹ and —XOXR¹⁹; wherein any aryl, heteroaryl, cycloalkyl or heterocycloalkyl substituent is further optionally substituted with 1 to 3 radicals independently selected from halo, nitro, cyano, C₁₋₆alkyl, C₁₋₆alkoxy, C₁₋₆alkylthio, hydroxy-C₁₋₆alkyl, halo-substituted-C₁₋₆alkyl and halo-substituted-C₁₋₆alkoxy; wherein X is a bond or C₁₋₄alkylene; R¹⁷ is selected from hydrogen and C₁₋₆alkyl; and R¹⁹ is selected from C₃₋₁₂cycloalkyl, C₃₋₈heterocycloalkyl, C₆₋₁₀aryl and C₅₋₁₀heteroaryl; wherein any aryl, heteroaryl, cycloalkyl or heterocycloalkyl of R¹⁹ is optionally substituted with 1 to 3 radicals independently selected from halo, nitro, cyano, C₁₋₆alkyl, C₁₋₆alkoxy, halo-substituted-C₁₋₆alkyl and halo-substituted-C₁₋₆alkoxy; and the N-oxide derivatives, prodrug derivatives, protected derivatives, individual isomers and mixture of isomers thereof; and the pharmaceutically acceptable salts and solvates (e.g. hydrates) of such compounds.

In a second aspect, the present invention provides a pharmaceutical composition that contains a compound of Formula I or a N-oxide derivative, individual isomers and mixture of isomers thereof; or a pharmaceutically acceptable salt thereof, in admixture with one or more suitable excipients.

In a third aspect, the present invention provides a method of treating a disease in an animal in which modulation of PPAR activity, particularly PPARδ, can prevent, inhibit or ameliorate the pathology and/or symptomology of the diseases, which method comprises administering to the animal a therapeutically effective amount of a compound of Formula I or a N-oxide derivative, individual isomers and mixture of isomers thereof, or a pharmaceutically acceptable salt thereof.

In a fourth aspect, the present invention provides the use of a compound of Formula I in the manufacture of a medicament for treating a disease in an animal in which PPAR activity, particularly PPARδ activity contributes to the pathology and/or symptomology of the disease.

In a fifth aspect, the present invention provides a process for preparing compounds of Formula I and the N-oxide derivatives, prodrug derivatives, protected derivatives, individual isomers and mixture of isomers thereof, and the pharmaceutically acceptable salts thereof.

### DETAILED DESCRIPTION OF THE INVENTION

Definitions

“Alkyl” as a group and as a structural element of other groups, for example halo-substituted-alkyl and alkoxy, can be either straight-chained or branched. C₁₋₆alkoxy includes, methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl can be phenyl or naphthyl, preferably phenyl. “Arylene” means a divalent radical derived from an aryl group.

“Heteroaryl” is as defined for aryl where one or more of the ring members are a heteroatom. For example heteroaryl includes pyridyl, indolyl, indazolyl, quinoxalinyl, quinolinyl, benzofuranyl, benzopyranyl, benzothiopyranyl, benzo[L1,3]dioxole, imidazolyl, benzo-imidazolyl, pyrimidinyl, furanyl, oxazolyl, isoxazolyl, triazolyl, tetrazolyl, pyrazolyl, thienyl, etc. “C₆₋₁₀arylC₀₋₄alkyl” means an aryl as described above connected via a alkylene grouping. For example, C₆₋₁₀arylC₀₋₄alkyl includes phenethyl, benzyl, etc.

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc.

“Heterocycloalkyl” means cycloalkyl, as defined in this application, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group. For example, C₃₋₈heterocycloalkyl as used in this application to describe compounds of the invention includes morpholino, pyrrolidinyl, piperazinyl, piperidinyl, piperidinylone, 1,4-dioxa-8-aza-spiro[4,5]dec-8-yl, etc.

“Halogen” (or halo) preferably represents chloro or fluoro, but can also be bromo or iodo.

“Treat”, “treating” and “treatment” refer to a method of alleviating or abating a disease and/or its attendant symptoms.

### DESCRIPTION OF THE PREFERRED EMBODIMENTS

The present invention provides compounds, compositions and methods for the treatment of diseases in which modulation of PPARδ activity can prevent, inhibit or ameliorate the pathology and/or symptomology of the diseases, which method comprises administering to the animal a therapeutically effective amount of a compound of Formula I.

In one embodiment, with reference to compounds of Formula I, p is an integer selected from 0 to 3; L² is selected from —XOX—, —XS(O)₀₋₂X— and —XS(O)₀₋₂XO—; wherein X is independently selected from a bond and C₁₋₄alkylene; wherein any alkylene of L² can be optionally substituted by 1 to 3 radicals selected from halo, C₁₋₆alkyl, C₁₋₆alkoxy, halo-substituted-C₁₋₆alkyl and halo-substituted-C₁₋₆alkoxy; and R¹³ is C₁₋₆alkyl, C₁₋₆alkoxy and halo.

In a further embodiment, R¹⁴ is selected from —XOXC(O)OR⁷ and —XC(O)OR¹⁷; wherein X is a bond or C₁₋₄alkylene; and R¹⁷ is selected from hydrogen and C₁₋₆alkyl; R¹⁵ and R¹⁶ are independently selected from —R¹⁸ and —YR¹⁸; wherein Y is a selected from C₁₋₆alkylene, C₂₋₆alkenylene, —C(O)NR¹⁷— and —OX—; X is a bond or C₁₋₄alkylene; R¹⁷ is selected from hydrogen and C₁₋₆alkyl; and R¹⁸ is selected from C₆₋₁₀aryl, C₃₋₁₂cycloalkyl and C₅₋₁₃heteroaryl; or R¹⁵ and R¹⁶ together with the atoms to which R¹⁵ and R¹⁶ are attached form fused bicyclic or tricyclic C₅₋₁₄heteroaryl; wherein any aryl, heteroaryl and cycloalkyl of R¹⁸, or the combination of R¹⁵ and R¹⁶, is optionally substituted with 1 to 3 radicals independently selected from halo, nitro, cyano, C₁₋₆alkyl, C₁₋₆alkoxy, C₁₋₆alkylthio, hydroxy-C₁₋₆alkyl, halo-substituted-C₁₋₆alkyl, halo-substituted-C₁₋₆alkoxy, C₃₋₁₂cycloalkyl, C₃₋₈heterocycloalkyl, C₆₋₁₀aryl optionally substituted with C₁₋₆alkoxy, C₅₋₁₃heteroaryl, —XS(O)O-₂R⁷, —XS(O)₀₋₂XR¹⁹—XNR¹⁷R¹⁷, —XNR¹⁷S(O)₀₋₂R¹⁷, —XN¹⁷C(O)R¹⁷, —XC(O)NR¹⁷R¹⁷, —XNR¹⁷C(O)R¹⁹, —XC(O)NR¹⁷R¹⁹, —XC(O)R¹⁹, —XNR¹⁷XR¹⁹ and —XOXR¹⁹; wherein X is a bond or C₁₋₄alkylene; R¹⁷ is selected from hydrogen and C₁₋₆alkyl; and R¹⁹ is selected from C₆₋₁₀aryl, C₅₋₁₀heteroaryl, C₃₋₈heterocycloalkyl and C₃₋₁₂cycloalkyl; wherein any aryl, heteroaryl, cycloalkyl or heterocycloalkyl of R¹⁹ is optionally substituted with 1 to 3 radicals independently selected from halo, nitro, cyano, C₁₋₆alkyl, C₁₋₆alkoxy, halo-substituted-C₁₋₆alkyl and halo-substituted-C₁₋₆alkoxy.

In a further embodiment, the invention provides a compound of Formula Ia:

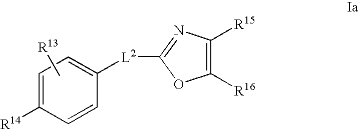

in which: L² is selected from —S(O)₀₋₂(CH₂)₁₋₄O—, —O(CH₂)₁₋₄S(O)₀₋₂₋, —CH₂S(O)O₀₋₂—, —S(O)₀₋₂CH₂—, —S(O)₀₋₂—, —CH₂O and —OCH₂—; R¹³ is selected from C₁₋₆alkyl, C₁₋₆alkoxy and halo; R¹⁴ is selected from —OCH₂C(O)OH and —CH₂C(O)OH; R¹⁵ and R¹⁶ are independently selected from —R¹⁸ and —YR¹⁸; wherein Y is selected from C₁₋₆alkylene, C₂₋₆alkenylene, —C(O)NH— and —O(CH₂)₁₋₃—; and R¹⁸ is selected from phenyl, biphenyl, cyclohexyl, naphthyl, benzo[1,3]dioxol-5-yl, benzo[b]furanyl, pyridinyl, pyrimidinyl, dibenzo-furan-2-yl, furanyl, benzo[b]thiophene, thiophenyl, phenoxathiin-4-yl, benzoxazolyl, 3-oxo-3,4-dihydro-2H-benzo[1,4]oxazin-6-yl, 2-oxo-2,3-dihydro-benzooxazol-6-yl, 2,3-dihydro-benzo[1,4]dioxin-6-yl, benzoxazolyl, 3,4-dihydro-2H-benzo[b][1,4]dioxepin-7-yl and quinolinyl; or R¹⁵ and R¹⁶ together with the atoms to which R¹⁵ and R¹⁶ are attached form 4,5-dihydro-naphtho[1,2-d]thiazol-2-yl, 4H-chromeno[4,3-d]thiazol-2-yl, 5,6-dihydro-4H-3-thia-1-aza-benzo[e]azulen-2-yl, benzthiazolyl, benzoxazolyl and 1-oxa-3-aza-cyclopenta[α]naphthalen-2-yl;

wherein any aryl, heteroaryl, cycloalkyl and heterocycloalkyl of R⁵, R¹⁶ or the combination of R¹⁵ and R¹⁶, is optionally substituted with 1 to 3 radicals independently selected from halo, cyano, nitro, methyl, isopropyl, isopropyl-sulfanyl, isopropyloxy, hydroxy-methyl, methyl-sulfanyl, methoxy, ethoxy, pentafluoroethoxy, trifluoromethyl, trifluoromethoxy, trifluoromethyl-sulfonyl, morpholino, phenoxy, benzoxy, ethyl-sulfonyl, dimethylamino, methyl-sulfonyl-amino, ethyl-sulfonyl, propyl, vinyl, propyloxy, sec-butoxy, trifluoromethyl-sulfanyl, dimethyl-amino-carbonyl, diethyl-amino-carbonyl, methyl-carbonyl-amino, methyl-carbonyl, cyclopentyl-oxy, isopropyl-methylamino-carbonyl, cyclopropyl-amino-carbonyl, cyclohexyl, morpholino, piperidinyl, indolyl, pyrrolidinyl, pyrrolidinyl-carbonyl, 2,3-dihydro-benzofuran-5-yl piperidinyl-carbonyl, morpholino-carbonyl, isopropyl-methyl-amino, isopropyl-methyl-amino-carbonyl, diethyl-amino, and phenyl optionally substituted with methoxy.

In a further embodiment are compounds of Formula Ib:

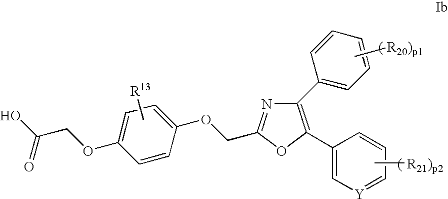

in which p1 and p2 are independently selected from 0, 1 and 2; Y is selected from N and CH; R¹³ is selected from C₁₋₆alkyl, C₁₋₆alkoxy and halo; R²⁰ is selected from trifluoromethyl and trifluoromethoxy; and R²′ is selected from isopropyloxy and methoxy.

Preferred compounds of Formula I are detailed in the Examples, infra. A preferred compound of the invention is {4-[4-(6-isopropoxy-pyridin-3-yl)-5-(4-trifluoromethoxy-phenyl)-oxazol-2-ylmethoxy]-2-methyl-phenoxy}-acetic acid.

Pharmacology and Utility

Compounds of the invention modulate the activity of PPARs and, as such, are useful for treating diseases or disorders in which PPARs contributes to the pathology and/or symptomology of the disease. This invention further provides compounds of this invention for use in the preparation of medicaments for the treatment of diseases or disorders in which PPARs, particularly PPARδ, contributes to the pathology and/or symptomology of the disease.

Such compounds may therefore be employed for the treatment of prophylaxis, dyslipidemia, hyperlipidemia, hypercholesteremia, atherosclerosis, atherogenesis, hypertriglyceridemia, heart failure, hyper cholesteremia, myocardial infarction, vascular diseases, cardiovascular diseases, hypertension, obesity, cachexia, HIV wasting syndrome, inflammation, arthritis, cancer, Alzheimer's disease, anorexia, anorexia nervosa, bulimia, skin disorders, respiratory diseases, ophthahnic disorders, IBDs (irritable bowel disease), ulcerative colitis and Crohn's disease. Preferably for the treatment of prophylaxis, dyslipidemia, hyperlipidemia, hypercholesteremia, atherosclerosis, atherogenesis, hypertriglyceridemia, cardiovascular diseases, hypertension, obesity, inflammation, cancer, skin disorders, IBDs (irritable bowel disease), ulcerative colitis and Crohn's disease.

Compounds of the invention can also be employed to treat long term critical illness, increase muscle mass and/or muscle strength, increase lean body mass, maintain muscle strength and function in the elderly, enhance muscle endurance and muscle function, and reverse or prevent frailty in the elderly.

Further, the compounds of the present invention may be employed in mammals as hypoglycemic agents for the treatment and prevention of conditions in which impaired glucose tolerance, hyperglycemia and insulin resistance are implicated, such as type-1 and type-2 diabetes, Impaired Glucose Metabolism (IGM), Impaired Glucose Tolerance (IGT), Impaired Fasting Glucose (IFG), and Syndrome X. Preferably type-1 and type-2 diabetes, Impaired Glucose Metabolism (IGM), Impaired Glucose Tolerance (IGT) and Impaired Fasting Glucose (IFG).

In accordance with the foregoing, the present invention further provides a method for preventing or treating any of the diseases or disorders described above in a subject in need of such treatment, which method comprises administering to said subject a therapeutically effective amount (See, “Administration and Pharmaceutical Compositions”, infra) of a compound of the invention or a pharmaceutically acceptable salt thereof. For any of the above uses, the required dosage will vary depending on the mode of administration, the particular condition to be treated and the effect desired. The present invention also concerns: i) a compound of the invention or a pharmaceutically acceptable salt thereof for use as a medicament; and ii) the use of a compound of the invention or a pharmaceutically acceptable salt thereof for the manufacture of a medicament for preventing or treating any of the diseases or disorders described above.

Administration and Pharmaceutical Compositions

In general, compounds of the invention will be administered in therapeutically effective amounts via any of the usual and acceptable modes known in the art, either singly or in combination with one or more therapeutic agents. A therapeutically effective amount can vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrollidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions can be sterilized and/or contain adjuvants, such as preserving, stabilizing, wetting or emulsifying agents, solution promoters, salts for regulating the osmotic pressure and/or buffers. In addition, they can also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations can also be used. Suitable formulations for topical application, e.g., to the skin and eyes, are preferably aqueous solutions, ointments, creams or gels well-known in the art. Such can contain solubilizers, stabilizers, tonicity enhancing agents, buffers and preservatives.

This invention also concerns a pharmaceutical composition comprising a therapeutically effective amount of a compound as described herein in combination with one or more pharmaceutically acceptable carriers.

Compounds of the invention can be administered in therapeutically effective amounts in combination with one or more therapeutic agents (pharmaceutical combinations).

Thus, the present invention also relates to pharmaceutical combinations, such as a combined preparation or pharmaceutical composition (fixed combination), comprising: 1) a compound of the invention as defined above or a pharmaceutical acceptable salt thereof; and 2) at least one active ingredient selected from:

a) anti-diabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; insulin sensitizer such as protein tyrosine phosphatase-1B (PTP-1B) inhibitors such as PTP-112; GSK3 (glycogen synthase kinase-3) inhibitors such as SB-517955, SB-4195052, SB-216763, N,N-57-05441 and N,N-57-05445; RXR ligands such as GW-0791 and AGN-194204; sodium-dependent glucose co-transporter inhibitors such as T-1095; glycogen phosphorylase A inhibitors such as BAY R3401; biguamides such as metformin; alpha-glucosidase inhibitors such as acarbose; GLP-1 (glucagon like peptide-1), GLP-1 analogs such as Exendin-4 and GLP-1 mimetics; DPPIV (dipeptidyl peptidase IV) inhibitors such as DPP728, LAF237 (vildagliptin—Example 1 of WO 00/34241), MK-0431, saxagliptin, GSK23A; an AGE breaker; a thiazolidone derivative (glitazone) such as pioglitazone, rosiglitazone, or (R)-1-{4-[5-methyl-2-(4-trifluoromethyl-phenyl)-oxazol-4-ylmethoxy]-benzenesulfonyl}-2,3-dihydro-1H-indole-2-carboxylic acid described in the patent application WO 03/043985, as compound 19 of Example 4, a non-glitazone type PPARγ agonist e.g. GI-262570;

b) hypolipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin;

c) an anti-obesity agent or appetite regulating agent such as phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine or cannabinoid receptor antagonists;

d) anti-hypertensive agents, e.g., loop diuretics such as ethacrynic acid, furosemide and torsemide; diuretics such as thiazide derivatives, chlorithiazide, hydrochlorothiazide, amiloride; angiotensin converting enzyme (ACE) inhibitors such as benazepril, captopril, enalapril, fosinopril, lisinopril, moexipril, perinodopril, quinapril, ramipril and trandolapril; inhibitors of the Na—K-ATPase membrane pump such as digoxin; neutralendopeptidase (NEP) inhibitors e.g. thiorphan, terteo-thiorphan, SQ29072; ECE inhibitors e.g. SLV306; ACE/NEP inhibitors such as omapatrilat, sampatrilat and fasidotril; angiotensin II antagonists such as candesartan, eprosartan, irbesartan, losartan, telmisartan and valsartan, in particular valsartan; renin inhibitors such as aliskiren, terlakiren, ditekiren, RO 66-1132, RO-66-1168; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors;

e) a HDL increasing compound;

f) Cholesterol absorption modulator such as Zetiag and KT6-971;

g) Apo-A1 analogues and mimetics;

h) thrombin inhibitors such as Ximelagatran;

i) aldosterone inhibitors such as anastrazole, fadrazole, eplerenone;

j) Inhibitors of platelet aggregation such as aspirin, clopidogrel bisulfate;

k) estrogen, testosterone, a selective estrogen receptor modulator, a selective androgen receptor modulator;

l) a chemotherapeutic agent such as aromatase inhibitors e.g. femara, anti-estrogens, topoisomerase I inhibitors, topoisomerase II inhibitors, microtubule active agents, alkylating agents, antineoplastic antimetabolites, platin compounds, compounds decreasing the protein kinase activity such as a PDGF receptor tyrosine kinase inhibitor preferably Imatinib ({N-{5-[4-(4-methyl-piperazino-methyl)-benzoylamido]-2-methylphenyl}-4-(3-pyridyl)-2-pyrimidine-amine}) described in the European patent application EP-A-0 564 409 as example 21 or 4-Methyl-N-[3-(4-methyl-imidazol-1-yl)-5-trifluoromethyl-phenyl]-3-(4-pyridin-3-yl-pyrimidin-2-ylamino)-benzamide described in the patent application WO 04/005281 as example 92; and

m) an agent interacting with a 5-HT₃ receptor and/or an agent interacting with 5-HT₄ receptor such as tegaserod described in the U.S. Pat. No. 5,510,353 as example 13, tegaserod hydrogen maleate, cisapride, cilansetron;

or, in each case a pharmaceutically acceptable salt thereof; and optionally a pharmaceutically acceptable carrier.

Most preferred combination partners are tegaserod, imatinib, vildagliptin, metformin, a thiazolidone derivative (glitazone) such as pioglitazone, rosiglitazone, or (R)-1-{4-[5-methyl-2-(4-trifluoromethyl-phenyl)-oxazol-4-ylmethoxy]-benzenesulfonyl}-2,3-dihydro-1H-indole-2-carboxylic acid, a sulfonylurea receptor ligand, aliskiren, valsartan, orlistat or a statin such as pitavastatin, simvastatin, fluvastatin or pravastatin.

Preferably the pharmaceutical combinations contains a therapeutically effective amount of a compound of the invention as defined above, in a combination with a therapeutically effective amount of another therapeutic agent as described above, e.g., each at an effective therapeutic dose as reported in the art. Combination partners (1) and (2) can be administered together, one after the other or separately in one combined unit dosage form or in two separate unit dosage forms. The unit dosage form may also be a fixed combination.

The structure of the active agents identified by generic or trade names may be taken from the actual edition of the standard compendium “The Merck Index” or the Physician's Desk Reference or from databases, e.g. Patents International (e.g. IMS World Publications) or Current Drugs. The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

In another preferred aspect the invention concerns a pharmaceutical composition (fixed combination) comprising a therapeutically effective amount of a compound as described herein, in combination with a therapeutically effective amount of at least one active ingredient selected from the above described group a) to m), or, in each case a pharmaceutically acceptable salt thereof.

A pharmaceutical composition or combination as described herein for the manufacture of a medicament for the treatment of for the treatment of dyslipidemia, hyperlipidemia, hypercholesteremia, atherosclerosis, hypertriglyceridemia, heart failure, myocardial infarction, vascular diseases, cardiovascular diseases, hypertension, obesity, inflammation, arthritis, cancer, Alzheimer's disease, skin disorders, respiratory diseases, ophthalmic disorders, inflammatory bowel diseases, EBDs (irritable bowel disease), ulcerative colitis, Crohn's disease, conditions in which impaired glucose tolerance, hyperglycemia and insulin resistance are implicated, such as type-1 and type-2 diabetes, Impaired Glucose Metabolism (IGM), Impaired Glucose Tolerance (IGT), Impaired Fasting Glucose (IFG), and Syndrome-X.

Such therapeutic agents include estrogen, testosterone, a selective estrogen receptor modulator, a selective androgen receptor modulator, insulin, insulin derivatives and mimetics; insulin secretagogues such as the sulfonylureas, e.g., Glipizide and Amaryl; insulinotropic sulfonylurea receptor ligands, such as meglitinides, e.g., nateglinide and repaglinide; insulin sensitizers, such as protein tyrosine phosphatase-1B (PTP-1B) inhibitors, GSK3 (glycogen synthase kinase-3) inhibitors or RXR ligands; biguamides, such as metformin; alpha-glucosidase inhibitors, such as acarbose; GLP-1 (glucagon like peptide-1), GLP-1 analogs, such as Exendin-4, and GLP-1 mimetics; DPPIV (dipeptidyl peptidase IV) inhibitors, e.g. isoleucin-thiazolidide; DPP728 and LAF237, hypolipidemic agents, such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin, fluindostatin and rivastatin, squalene synthase inhibitors or FXR (liver X receptor) and LXR (farnesoid X receptor) ligands, cholestyramine, fibrates, nicotinic acid and aspirin. A compound of the present invention may be administered either simultaneously, before or after the other active ingredient, either separately by the same or different route of administration or together in the same pharmaceutical formulation.

The invention also provides for pharmaceutical combinations, e.g. a kit, comprising: a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

Processes for Making Compounds of the Invention

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

Compounds of Formula I, in which R¹⁵ is cyclic (e.g. cycloalkyl, heterocycloalkyl, aryl and heteroaryl), can be prepared by proceeding as in reaction scheme

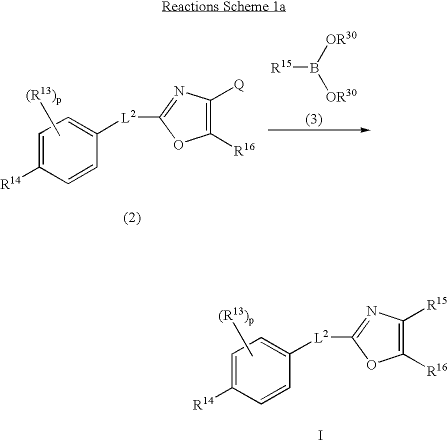

in which p, R¹³, R¹⁴, R¹⁶ and L² are as defined for Formula I in the Summary of the Invention. Q is a halogen, preferably Cl or Br; and R³⁰ is independently selected from hydrogen, C₁₋₆alkyl or the R³⁰ radicals can be cyclized. Compounds of Formula I are prepared by reacting a compound of formula 2 with a compound of formula 3 in the presence of a suitable catalyst (e.g., Pd(Ph₃)₄, or the like), a suitable base (e.g., Na₂CO₃, or the like) and a suitable solvent (e.g., water, ethanol, DME or the like). The reaction is carried out in the temperature range of about 120 to about 200° C. (microwave) and takes up to about 20 minutes to complete.

Compounds of Formula I, in which R¹⁶ is cyclic (e.g. cycloalkyl, heterocycloalkyl, aryl and heteroaryl), can be prepared by proceeding as in reaction scheme Ib:

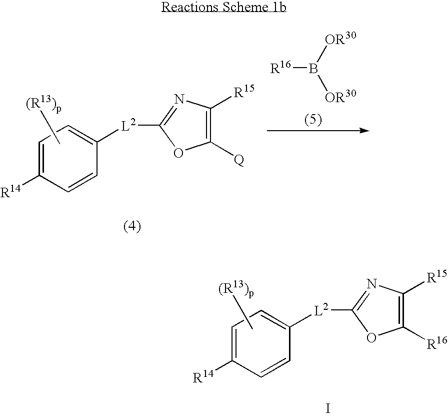

in which p, R¹³, R¹⁴, R¹⁶ and L² are as defined for Formula I in the Summary of the Invention. Q is a halogen, preferably Cl or Br; and R³⁰ is independently selected from hydrogen, C₁₋₆alkyl or the R³⁰ radicals can be cyclized. Compounds of Formula I are prepared by reacting a compound of formula 4 with a compound of formula 5 in the presence of a suitable catalyst (e.g., Pd(Ph₃)₄, or the like), a suitable base (e.g., Na₂CO₃, or the like) and a suitable solvent (e.g., water, ethanol, DME or the like). The reaction is carried out in the temperature range of about 120 to about 200° C. (microwave) and takes up to about 20 minutes to complete.

Compounds of Formula I, in which R¹⁴ is defined by —Y—COOR³¹, can be prepared by proceeding as in reaction scheme 2:

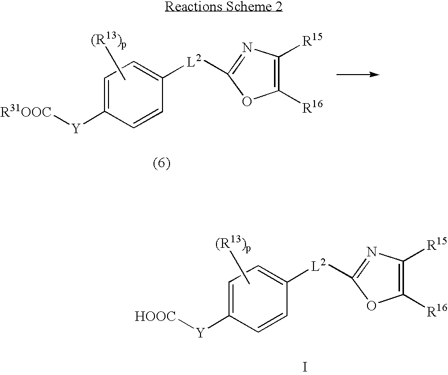

in which p, R¹³, R¹⁵, R¹⁶ and L² are as defined for Formula I in the Summary of the Invention; Y is —XOX— or —X— (wherein X is independently selected from a bond or C₁₋₄alkylene as defined in the Summary of the Invention) and R³¹ is an alkyl group, for example, methyl. Compounds of Formula I are prepared by reacting a compound of formula 4 in the presence of a suitable base (e.g., lithium hydroxide, or the like) and a suitable solvent (e.g., THF, water or the like). The reaction is carried out in the temperature range of about 0 to about 50° C. and takes up to about 30 hours to complete.

Compounds of Formula 9, in which R³ is —CH₃, —SH, —C(O)OC₂H₅, —CH₂OC(O)C(CH₃)₃ or a group defined by:

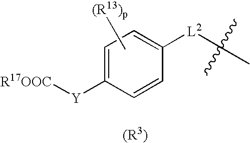

wherein Y is —XOX— or —X—; and P, R¹³, L², X and R¹⁷ are as Defined in the Summary of the Invention), can be prepared by proceeding as in reaction scheme 3:

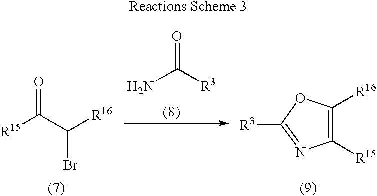

in which p, R¹³, R¹⁷ and L² are as defined for Formula I in the Summary of the Invention; R¹⁵ and R¹⁶ independently are selected from hydrogen, alkyl or any cyclic radical (cycloalkyl, heterocycloalkyl, aryl and heteroaryl as defined in the Summary of the Invention). Compounds of Formula 9 are prepared by reacting a compound of formula 7 with a compound of formula 8 optionally in the presence of a solvent (e.g., ethanol, or the like). The reaction is carried out in the temperature range of about 10 to about 200° C. and takes up to about 30 hours to complete.

Compounds of Formula I can be prepared by proceeding as in reaction scheme 4a and 4b:

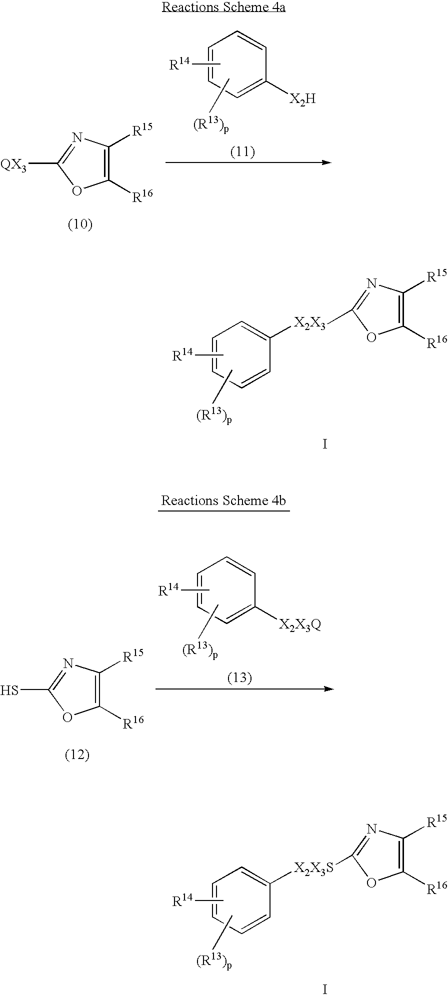

in which p, R¹³, R¹⁴, R¹⁵ and R¹⁶ are as defined for Formula I in the Summary of the Invention; X₂ is S or O; X₃ is a bond or C₁₋₄alkylene; and Q is a halo group, preferably Br or Cl. Compounds of Formula I are prepared by reacting a compound of formula 10 with a compound of formula 11 or a compound of formula 12 with a compound of formula 13 in the presence of a suitable solvent (e.g., cyanomethyl, ethanol or the like). The reaction is carried out in the temperature range of about 10 to about 80° C. and takes up to about 24 hours to complete.

Compounds of Formula I can be prepared by proceeding as in reaction scheme 5:

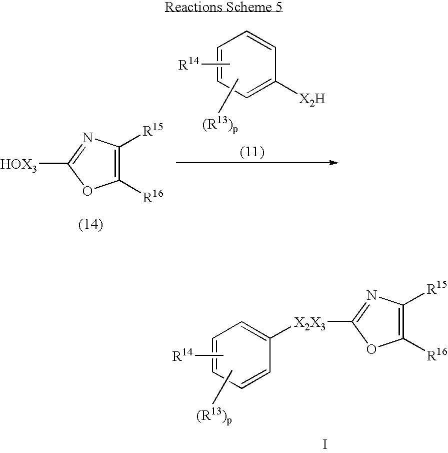

in which p, R¹³, R¹⁴, R¹⁵ and R¹⁶ are as defined for Formula I in the Summary of the Invention; X₂ is S or O; and X₃ is a bond or C₁₋₄alkylene. Compounds of Formula I are prepared by reacting a compound of formula 14 with a compound of formula 11 in the presence of a suitable solvent (e.g., DCM, THF or the like) and a suitable activating reagent (e.g., triphenylphosphine, diethylazodicarboxylate or the like). The reaction is carried out in the temperature range of about 0 to about 50° C. and takes up to about 24 hours to complete.

Detailed reaction conditions are described in the examples, infra.

Additional Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base. Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, the compounds of Formula I can be made by a process, which involves:

(a) that of reaction scheme 1a, 1b, 2, 3, 4a, 4b or 5; and

(b) optionally converting a compound of the invention into a pharmaceutically acceptable salt;

(c) optionally converting a salt form of a compound of the invention to a non-salt form;

(d) optionally converting an unoxidized form of a compound of the invention into a pharmaceutically acceptable N-oxide;

(e) optionally converting an N-oxide form of a compound of the invention to its unoxidized form;

(f) optionally resolving an individual isomer of a compound of the invention from a mixture of isomers;

(g) optionally converting a non-derivatized compound of the invention into a pharmaceutically acceptable prodrug derivative; and

(h) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

### EXAMPLES

The present invention is further exemplified, but not limited, by the following intermediates and examples that illustrate the preparation of compounds of Formula I according to the invention.

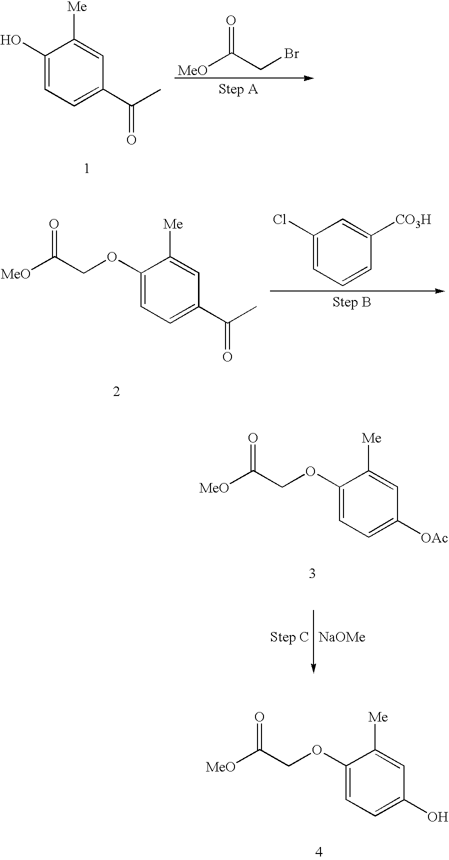

### Intermediate 4

### (4-Hydroxy-2-methyl-phenoxy)-acetic acid methyl ester

Step A: 4′-Hydroxy-3′-methylacetophenone 1 (25 g, 166.4 mmol) and methyl-bromoacetate (25.5 g, 166.4 mmol) was dissolved in MeCN (600 mL). Cs₂CO₃ (117.8 g, 332.9 mmol) was added and the mixture was stirred overnight at rt. After insoluble salts were filtered and washed with MeCN, the solvent was removed and the remainder was taken up in EtOAc and washed subsequently with 1 M HCl (3×500 mL) and H₂O (2×500 mL). The organic layer was dried (MgSO₄), filtered and concentrated to afford 2 (35.9 g, 161.4 mmol, 97%) as a white solid. I

Step B: (4-Acetyl-2-methyl-phenoxy)-acetic acid methyl ester 2 (33 g, 151.3 mmol), 77% mCPBA (54.9 g, 264.8 mmol) and p-TsOH (2.9 g, 15.1 mmol) in DCM (650 mL) were heated under reflux for 48 h. The reaction mixture was then washed with 1 M KI (2×500 mL) and NaHSO₃ (2×500 mL). The organic layer was dried (MgSO₄), filtered and concentrated to afford 3 (28.8 g, 121.0 mmol, 80%) as a brown syrup.

Step C: A solution of (4-acetoxy-2-methyl-phenoxy)-acetic acid methyl ester 3 (25 g, 105.0 mmol) in dry MeOH (400 mL) was combined with a 0.5 M solution of NaOMe in MeOH (210 mL, 105.0 mmol) and stirred for 1 h at rt. The solution was neutralized with 1 M HCl and washed with H₂O (2×500 mL). The organic layer was dried (MgSO₄), filtered and concentrated to afford 4 (17.5 g, 89.3 mmol, 85%) as a brown solid:

¹H-NMR (400 MHz, CD₃OD) δ=6.65-6.51 (m, 3H), 4.60 (s, 2H), 3.75 (s, 3H), 2.19 (s, 3H). MS calcd. for C₁₀H₁₃O₄ (M+H⁺) 197.1, found 197.2.

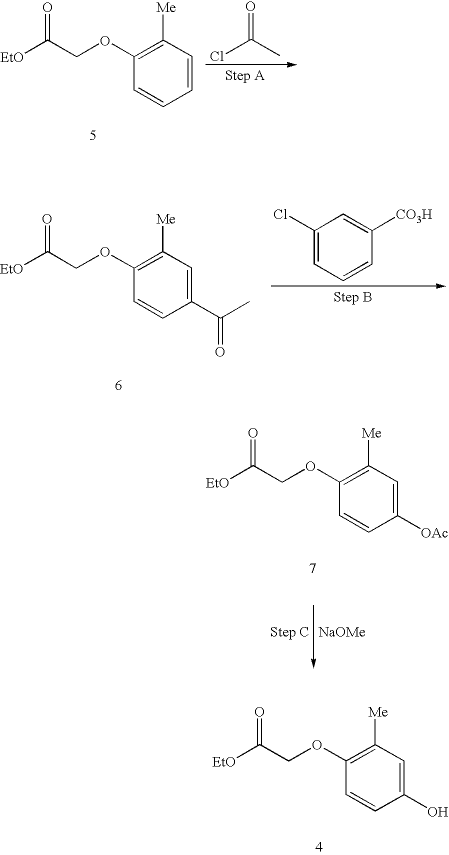

### Intermediate 4 (Alternative Route)

### (4-Hydroxy-2-methyl-phenoxy)-acetic acid methyl ester

Step A: (2-Methylphenoxy)acetic acid ethyl ester 5 (66.03 g, 340 mmol) was dissolved in dichloroethane (400 mL). Aluminum chloride (100.02 g, 750 mmol, 2.2 equiv.) was added and the light-brown mixture was stirred for 10 minutes at room temperature until homogenous. Acetyl chloride (35 mL, 493 mmol, 1.45 equiv.) was added dropwise using an addition funnel. The rate of addition was adjusted to maintain a relatively slow emission of hydrogen chloride gas. The resulting dark brown solution was allowed to cool off to room temperature, then was poured over 300 g of crushed ice. The mixture was diluted with 300 mL dichloromethane and washed successively with water, saturated NaHCO₃ solution, water, saturated NH₄Cl solution, and brine. The organic layer was dried over Na₂SO₄, filtered and concentrated to afford 6 (76.54 g, 324 mmol, 95%) as a brown oil that solidified as a crystalline mass. ¹H-NMR (400 MHz, CDCl₃) δ=7.79 (d, J=2.0 Hz, 1H), 7.77 (dd, J=2.0, 8.4 Hz, 1H), 6.69 (d, J=8.4 Hz, 1H), 4.71 (s, 2H), 4.26 (q, J=7.2 Hz, 2H), 2.54 (s, 3H), 2.32 (s, 2H), 1.29 (t, J=7.2 Hz, 3H).

Step B: (4-Acetyl-2-methyl-phenoxy)-acetic acid ethyl ester 6 (76.54 g, 324 mmol), 77% mCPBA (100.31 g, 407 mmol, 1.26 equiv.) and p-TsOH (13 g, 68 mmol, 21 mol %) in dichloroethane (450 mL) were heated to 50° C. for 30 h. The reaction mixture was then washed with 1 M KI (2×500 mL) and NaHSO₃ (2×500 mL). The organic layer was dried (MgSO₄), filtered and concentrated to afford 7 as a brown syrup.

Step C: A solution of (4-acetoxy-2-methyl-phenoxy)-acetic acid ethyl ester i (from step B above) in dry MeOH (400 mL) was combined with a 0.5 M solution of NaOMe in MeOH (650 mL, 325 mmol) and stirred for 2 h at rt. The solution was neutralized with 1 M HCl and washed with H₂O (2×500 mL). The organic layer was dried (Na₂SO₄), filtered and concentrated to afford 4 (21.7 g, 111 mmol, 34%, two steps) as a light-brown solid: ¹H-NMR (400 MHz, CDCl₃) δ=6.58 (d, J=2.8 Hz, 1H), 6.54 (d, J=8.4 Hz), 6.50 (dd, J=2.8, 8.4 Hz, 1H), 4.7 (br. s, 1H), 4.54 (s, 2H), 3.73 (s, 3H), 2.17 (s, 3H). MS calcd. for C₁₀H₁₃O₄ (M+H⁺) 197.1, found 197.4.

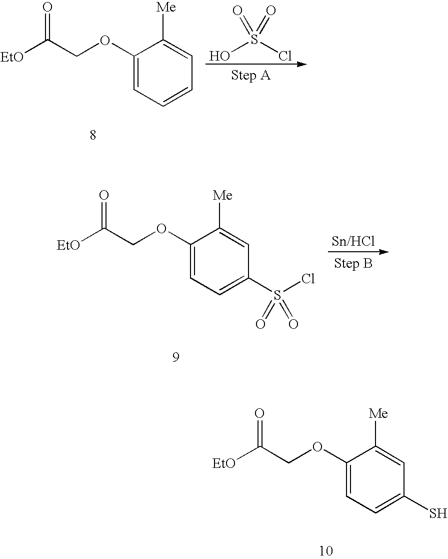

### Intermediate 10

### (4-Mercapto-2-methyl-phenoxy)-acetic acid ethyl ester

Step A: A 500 mL three-necked round bottom flasked was charged with chlorosulfonic acid (25 mL, 373.9 mmol), flushed with nitrogen and cooled to 0° C. Under nitrogen and vigorous stirring, ethyl (2-methylphenoxy)acetate 8 (40 g, 206.2 mmol) was added dropwise. The mixture was stirred at for 90 min at 0° C., then poured on ice-water (200 mL). After the mixture was stirred for an additional 45 min at rt, the white precipitate was filtered, washed with ice-water and dried in vacuo to afford 9 (28.4 g, 97.0 mmol, 47%) as a white solid.

Step B: (4-Chlorosulfonyl-2-methyl-phenoxy)-acetic acid ethyl ester 9 (25 g, 85.4 mmol) and tin (50.8 g, 427 mmol) were suspended in EtOH and cooled to 0° C. After a solution of 4 N HCl in dioxane (107 mL, 427 mmol) was added dropwise, the resulting mixture was heated to reflux for 3 h. Then the mixture was concentrated in vacuo, the remainder taken up in chloroform and filtered. The filtrate was concentrated in vacuo to a yellow oil, which was purified by chromatography (silica, Hex/EtOAc gradient) to afford 10 (15 g, 66.4 mmol, 78%) as a colourless oil: ¹H-NMR (400 MHz, CDCl₃) δ=7.14 (m, 1H), 7.07-7.10 (m, 1H), 6.59 (m, 1H), 4.60 (s, 2H), 4.25 (q, J=7.1 Hz, 2H), 3.33 (s, 1H), 2.24 (s, 3H), 1.29 (t, J=7.1 Hz, 3H). MS calcd. for ClH₁₄O₃S (M+H⁺) 227.1, found 227.4.

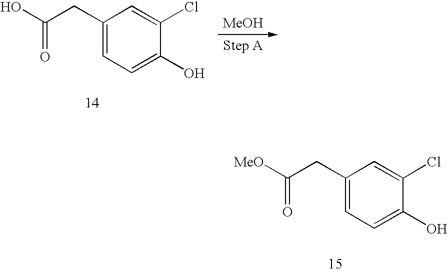

### Intermediate 15

### (3-Chloro-4-hydroxy-phenyl)-acetic acid methyl ester

Step A: 3-Chloro-4-hydroxy-phenyl)-acetic acid 14 (20 g, 107 mmol) was dissolved in MeOH (250 mL) containing catalytic amounts of conc. H₂SO₄ (2.5 mL). The solution was heated to reflux overnight. The solvent was evaporated, the remainder was dissolved in DCM and washed with H₂O (3×200 mL). The organic layer was dried (MgSO₄), filtered and concentrated to afford 15 (21.5 g, 107 mmol, 100%) as a light yellow solid: ¹H-NMR (400 MHz, CD₃OD) δ=7.21 (d, J=2.1 Hz, 1H), 7.01 (dd, J=2.1 Hz, J=8.3, 1H), 6.84 (d, J=8.3 Hz, 1H), 3.67 (s, 3H), 3.54 (s, 2H). MS calcd. for C₉H₁₀ClO₃ (M+H⁺) 201.0, found 201.2.

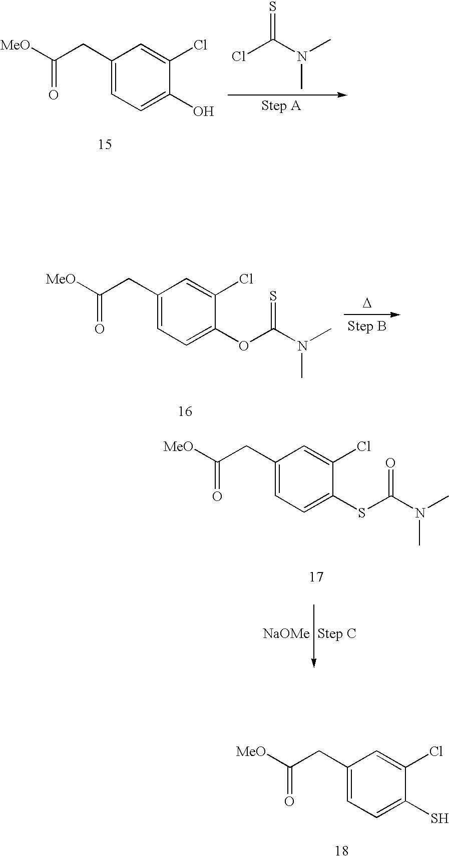

### Intermediate 18

### (3-Chloro-4-mercapto-phenyl)-acetic acid methyl ester

Step A: 3-(Chloro-4-hydroxy-phenyl)-acetic acid methyl ester 15 (4.1 g, 21.4 mmol), dimethyl thiocarbamoylchloride (3.2 g, 25.6 mmol), Et₃N (5.9 mL, 42.8 mmol) and DMAP (261 mg, 2.14 mmol) were dissolved in dry dioxane (30 mL) and heated to reflux for 16 h under nitrogen. The reaction mixture was cooled to rt, diluted with EtOAc and washed with H₂O (3×50 mL). The organic layer was dried (MgSO₄), filtered and concentrated to afford 16 (5.2 g, 18.1 mmol, 85%) as a colourless oil.

Step B: (3-Chloro-4-dimethylthiocarbamoyloxy-phenyl)-acetic acid methyl ester 16 (5.2 g, 18.1 mmol) was transferred to a 250 mL three-necked round bottom flask equipped with a thermometer. Tetradecane (45 mL) was added and the mixture was heated to reflux (250° C.) overnight. After cooling to rt the solvent was decanted, the remaining oil washed several times with hexanes and purified by chromatography (silica, Hex/EtOAc gradient) to afford 17 (3.1 g, 10.8 mmol, 60%) as a brown oil.

Step C: (3-Chloro-4-dimethylcarbamoylsulfanyl-phenyl)-acetic acid methyl ester 17 (3.1 g, 10.8 mmol) was dissolved in 0.5 M NaOMe solution. The mixture was heated to reflux for 4 h, then acidified with 1 M HCl. The organic solvent was evaporated, the remainder was extracted into EtOAc (50 mL) and washed with H₂O (2×50 mL). The organic layer was dried (MgSO₄), filtered, concentrated and purified (silica, hexanes/EtOAc gradient) to afford 18 (1.5 g, 6.9 mmol, 64%) as a pale yellow oil: ¹H-NMR (400 MHz, CDCl₃) δ=7.30-7.26 (m, 2H), 7.06-7.03 (m, 1H) 3.87 (s, 1H), 3.69 (s, 3H), 3.55 (s, 2H). MS calcd. for C₉H₁₀ClO₂S (M+H⁺) 217.0, found 217.3.

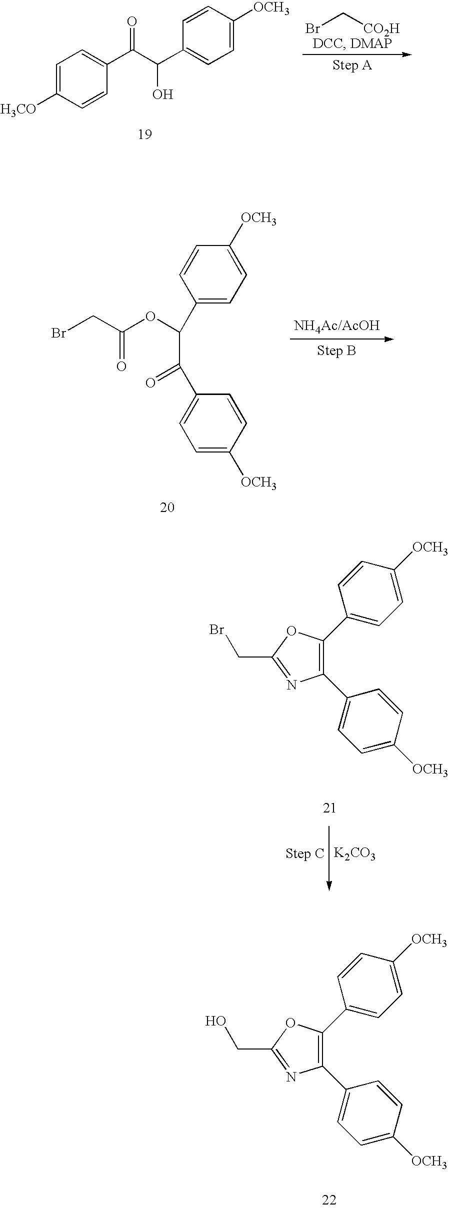

### Intermediate 22

### 2-Hydroxymethyl-4,5-bis-(4-methoxy-phenyl)-oxazole

Step A: A mixture of anisoin 19 (1.00 g, 3.49 mmol), bromoacetic acid (0.53 g, 3.84 mmol), 1,3-dicyclohexycarbodiimide (0.88 g, 4.23 mmol), DMAP (21.5 mg, 0.17 mmol) and CH₂Cl₂ (25 mL) was stirred at room temperature under an atmosphere of N₂. After 17 h, the mixture was filtered and concentrated to leave an oil, which was purified by flash chromatography. Elution with a mixture of hexane and ethyl acetate (10:1) afforded bromo-acetic acid 1,2-bis-(4-methoxy-phenyl)-2-oxo-ethyl ester 20 (1.02 g, 2.59 mmol, 74%) as a slightly yellow solid: ¹H-NMR (400 MHz, CDCl₃) δ=7.89 (d, J=8.4 Hz, 2H), 7.37 (d, J=8.8 Hz, 2H), 6.90-6.84 (m, 5H), 4.03-3.96 (m, 2H), 3.82 (s, 3H), 3.77 (s, 3H).

Step B: A solution of bromo-acetic acid 1,2-bis-(4-methoxy-phenyl)-2-oxo-ethyl ester 20 (393.0 mg, 1.00 mmol) and NH₄OAc (384.0 mg, 5.0 mmol) in AcOH (6 mL) was heated at reflux for 1.5 h. Then the mixture was poured onto H₂O and extracted with CH₂Cl₂ to leave an oil. Flash chromatography using a mixture of hexane and ethyl:acetate (10:1) as eluent afforded 21 (283.0 mg, 0.76 mmol, 76%) as a white solid: ¹H-NMR (400 MHz, CDCl₃) δ=7.58-7.50 (m, 4H), 6.90 (d, J=8.0 Hz, 4H), 5.22 (s, 2H), 3.83 (s, 6H).

Step C: A mixture of intermediate 21 (75.0 mg, 0.20 mmol), potassium carbonate (110.4 mg, 0.80 mmol) and CH₃CN (5 mL) was heated at reflux for 2 h. The mixture was poured onto H₂O, EtOAc (50 mL) was added. The organic layer was dried and filtered. The solvent was removed in vacuo to afford 2-hydroxymethyl-4,5-bis-(4-methoxy-phenyl)-oxazole 22 (50.0 mg, 0.16 mmol, 80%) as a white solid. %): ¹H-NMR (400 MHz, CDCl₃) δ=7.49 (d, J=8.8 Hz, 2H), 7.45 (d, J=8.8 Hz, 2H), 6.84 (d, J=5.2 Hz, 1H), 6.82 (d, J=5.2 Hz, 2H), 4.72 (s, 2H), 3.77 (s, 6H). MS calcd. for C₁₈H₁₈ NO₄ (M+H⁺) 312.12, found 312.10.

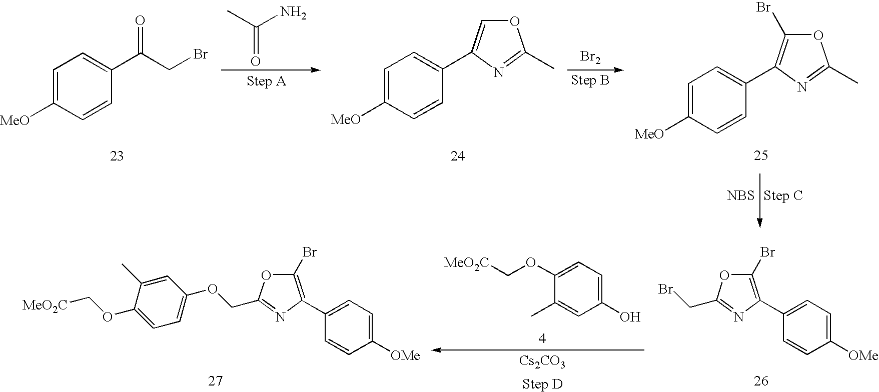

### Intermediate 27

### {4-[5-bromo-4-(4-methoxy-phenyl)-oxazol-2-ylmethoxy]-2-methyl-phenoxy}-acetic acid methyl ester

Step A: 2-Bromo-4′-methoxyacetophenone 23 (20.0 g, 87.3 mmol) and acetamide (15.5 g, 262.0 mmol) were heated to 150° C. for 2 hours. The mixture was cooled to rt, diluted with EtOAc and washed with saturated Na₂CO₃ and brine. The organic layer was dried (MgSO₄), filtered and concentrated to give crude product, which was purified by silica gel chromatography (EtOAc/hexane gradient) to give 4-(4-methoxy-phenyl)-2-methyl-oxazole 24 (10.3 g, 62%) as a white solid: ¹H-NMR (400 MHz, CDCl₃) δ=7.65 (s, 1H), 7.56 (d, J=8.8 Hz, 2H), 6.85 (d, J=8.8 Hz, 2H), 3.76 (s, 3H), 2.44 (s, 3H). MS calcd. for C₁₁H₁₂NO₂ (M+H⁺) 190.2, found 190.1.

Step B: 4-(4-Methoxy-phenyl)-2-methyl-oxazole 24 (212 mg, 1.12 mmol) was dissolved in carbon tetrachloride (10 mL), then bromine (63.3 μL, 1.23 mmol) was added and the mixture was stirred at rt for 30 min. The solid was collected by filtration, then dissolved in EtOAc (50 mL) and washed with saturated NaHCO₃ (20 mL) and brine (10 mL). The organic layer was dried (MgSO₄), filtered and concentrated to give 5-bromo-4-(4-methoxy-phenyl)-2-methyl-oxazole 25 as a white solid (240 mg, 80%): ¹H-NMR (400 MHz, CDCl₃) δ=7.78 (d, J=8.8 Hz, 2H), 6.88 (d, J=8.8 Hz, 2H), 3.77 (s, 3H), 2.43 (s, 3H). MS calcd. for C₁₁H, BrNO₂ (M+H⁺) 269.1, found 269.0.

Step C: N-bromosuccinimide (4.89 g, 27.5 mmol) was added to a solution of 5-bromo-4-(4-methoxy-phenyl)-2-methyl-oxazole 25 (6.7 g, 25.0 mmol) in carbon tetrachloride (250 mL). The above solution was stirred at room temperature for 15 hours. Then the mixture washed with saturated Na₂CO₃ and brine. The organic layer was dried (MgSO₄), filtered and concentrated to give crude product, which was purified by silic gel chromatography with hexane/ether (gradient) to give 5-bromo-2-bromomethyl-4-(4-methoxy-phenyl)-oxazole 26 (3.3 g, 38%) as a white solid: ¹H-NMR (400 MHz, CDCl₃) δ 7.87 (d, J=8.8 Hz, 2H), 6.97 (d, J=8.8 Hz, 2H), 4.46 (s, 2H), 3.85 (s, 3H). MS calcd. for C₁₁H₁₀Br₂NO₂ (+H⁺) 348.0, found 347.9.

Step D: A mixture of 5-bromo-2-bromomethyl-4-(4-methoxy-phenyl)-oxazole (2.75 g, 7.92 mmol) 26, (4-hydroxy-2-methyl-phenoxy)-acetic acid methyl ester 4 (1.24 g, 6.34 mmol) and Cs₂CO₃ (3.01 g, 9.48 mmol) in MeCN (200 mL) was stirred at rt for 1 h. The mixture was filtered, then concentrated to give crude product, which was purified by silic gel chromatography with EtOAc/hexane (gradient) to give {4-[5-bromo-4-(4-methoxy-phenyl)-oxazol-2-ylmethoxy]-2-methyl-phenoxy}-acetic acid methyl ester 27 (2.51 g, 86%) as a solid: ¹H-NMR (400 MHz, CDCl₃) δ=7.81 (d, J=9.8 Hz, 2H), 6.90 (d, J=9.8 Hz, 2H), 6.80 (d, J=3.2 Hz, 1H), 6.71 (m, 1H), 6.58 (m, 1H), 4.99 (s, 2H), 4.52 (s, 2H), 3.78 (s, 3H), 3.72 (s, 3H), 2.20 (s, 3H). MS calcd. for C₂₁H₂₁BrNO₆ (M+H⁺) 463.3, found 463.0.

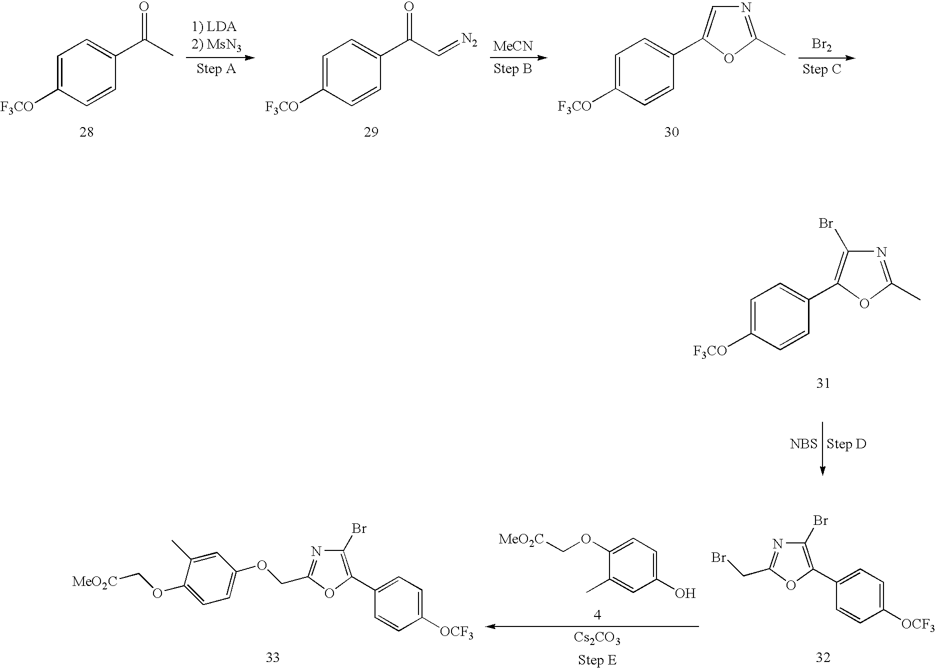

### Intermediate 33

### {4-[4-bromo-5-(4-trifluoromethoxy-phenyl)-oxazol-2-ylmethoxy]-2-methyl-phenoxy}-acetic acid methyl ester

Step A: 1,1,1,3,3,3-hexamethyldisilazane (8.93 g, 55.35 mmol) was dissolved in dry THF (50 mL) in a flame dried three-necked flask, and cooled to 0° C. n-Butyl lithium (2.5 M in hexanes, 21.55 mL, 53.88 mmol) was added dropwise. After stirring the resulting solution for 10 min at 0° C., it was cooled to −78° C. 4′-(trifluoromethoxy)acetophenone 28 (10.0 g, 48.98 mmol) dissolved in dry THF (64 mL) was added dropwise over 30 min. The reaction was kept stirring for 45 min at −78° C. 2,2,2-trifluoroethyltrifluoroacetate (11.43 g, 58.78 mmol) was added rapidly. After 20 min, the reaction was poured into a separation funnel containing 200 mL of 5% HCl and extracted with 250 mL diethyl ether. The organic layer washed with brine, dried over MgSO₄, and concentrated. The residue was dissolved in acetonitril (50 mL), then water (0.88 mL, 48.98 mmol) and triethylamine (7.43 g, 73.47 mmol) were added. Freshly prepared methanesulfonyl azide (8.98 g, 73.47 mmol) in a solution of acetonitrile (16 mL) was added over 30 min at room temperature. [Methanesulfonyl azide was prepared from the following procedure: methanesulfonyl chloride (8.85 g, 73.47 mmol) was dissolved in acetone (50 mL). Sodium azide (7.56 g, 116.0 mmol) was then added over 30 min. The reaction was stirred for 1.5 h at rt, then it was filtered, and washed with acetone. The mixture was concentrated to give crude product.] The reaction was kept stirring for 1 h, then concentrated. The residue was diluted with diethyl ether (200 mL), washed with 10% NaOH three times, and then with brine. It was dried over MgSO₄, filtered and concentrated to give crude product, which was purified by silica gel chromatography (ether/hexane, gradient) to give 2-diazo-4′-trifluoromethoxyacetophenone (29) (7.93 g, 70%) as a yellow solid: ¹H-NMR (400 MHz, CDCl₃) δ=7.82 (d, J=8.8 Hz, 2H), 7.29 (d, J=8.8 Hz, 2H), 5.89 (s, 1H). MS calcd. for C₉H₆F₃N₂O₂ (M⁺) 230.0, found 203.0 (M+H⁺—N₂).

Step B: Aluminum chloride (19.6 g, 146.78 mmol) was carefully added in portions into anhydrous acetonitrile (200 mL). 2-Diazo-4′-trifluoromethoxyacetophenone 29 (16.89 g, 73.39 mmol) dissolved in anhydrous acetonitrile (200 mL) was added by syringe dropwise over 30 min at rt with an outlet to release generated nitrogen. The reaction was stirred for 45 min, then it was poured into ditheyl ether (500 mL). The solution was carefully quenched with 0.2 N HCl. Then it was basified with 1 N NaOH to PH=9-10. The organic layer was separated. The aqueous layer was extracted twice with diethyl ether. The combined organic layers were washed with water and brine, dried (MgSO₄), filtered, and concentrated to give crude product, which was purified by silica gel chromatography (ether/hexane gradient) to give 2-methyl-5-(4-trifluoromethoxy-phenyl)-oxazole 30 (14.0 g, 78%) as an oil: ¹H-NMR (400 MHz, CDCl₃) δ=7.56 (d, J=8.8 Hz, 2H), 7.19 (d, J=8.8 Hz, 2H), 7.13 (s, 1H), 2.46 (s, 3H). MS calcd. for C₁₁H₉F₃NO₂ (M+H⁺) 244.1, found 244.0.

Step C: 2-Methyl-5-(4-trifluoromethoxy-phenyl)-oxazole 30 (3.07 g, 12.62 mmol) was dissolved in chloroform (100 mL), then bromine (648.7 μL, 12.62 mmol) was added dropwise and the mixture was stirred at rt for 15 h. The solution was diluted with CH₂Cl₂ (100 mL) and washed with saturated NaHCO₃ (150 mL) and brine (130 mL). The organic layer was dried (MgSO₄), filtered and concentrated to give crude product, which was purified by silic gel chromatography with ether/hexane (gradient) to give 4-bromo-2-methyl-5-(4-trifluoromethoxy-phenyl)-oxazole 31 as an oil (2.0 g, 49.4%): ¹H-NMR (400 MHz, CDCl₃) δ=7.86 (d, 2H, J=8.6 Hz), 7.22 (d, 2H, J=8.6 Hz), 2.47 (s, 3H). MS calcd. for C₁₁H₈BrF₃NO₂ (M+H⁺) 321.9, found 321.9.

Step D: N-bromosuccinimide (4.89 g, 27.5 mmol) was added to a solution of 4-bromo-2-methyl-5-(4-trifluoromethoxy-phenyl)-oxazole 31 (2.0 g, 6.25 mmol) in carbon tetrachloride (40 mL). The above solution was stirred at 75⁰ C. for 20 h. The solution was diluted with CH₂Cl₂ (100 mL) and washed with saturated aqueous Na₂CO₃ and brine. The organic layer was dried (MgSO₄), filtered and concentrated to give crude product, which was purified by silic gel chromatography with hexane/ether (gradient) to give 4-bromo-2-bromomethyl-5-(4-trifluoromethoxy-phenyl)-oxazole 32 (1.64 g, 66.0%) as a white solid:

¹H-NMR (400 MHz, CDCl₃) δ=7.91 (d, 2H, J=8.6 Hz), 7.25 (d, 2H, J=8.6 Hz), 4.41 (s, 3H). MS calcd. for C₁₁H₇Br₂F₃NO₂ (M+H⁺) 399.9, found 399.8.

Step E: A mixture of 4-bromo-2-bromomethyl-5-(4-trifluoromethoxy-phenyl)-oxazole 32 (895 mg, 2.232 mmol), (4-hydroxy-2-methyl-phenoxy)-acetic acid, methyl ester 4 (482 mg, 2.455 mmol) and Cs₂CO₃ (836 mg, 2.567 mmol) in MeCN (50 mL) was stirred at rt for 3 h. The mixture was filtered, then concentrated to give crude product, which was purified by silic gel chromatography with EtOAc/hexane (gradient) to give {4-[4-bromo-5-(4-trifluoromethoxy-phenyl)-oxazol-2-ylmethoxy]-2-methyl-phenoxy}-acetic acid methyl ester 33 (926 mg, 80.0%) as a solid: ¹H-NMR (400 MHz, CDCl₃) δ=7.89 (d, J=8.4 Hz, 2H), 7.23 (d, J=8.4 Hz, 2H), 6.79 (d, J=3.2 Hz, 1H), 6.70 (m, 1H), 6.59 (m, 1H), 5.03 (s, 2H), 4.53 (s, 2H), 3.72 (s, 3H), 3.72 (s, 3H), 2.21 (s, 3H). MS calcd. for C₂₁H₁₈BrF₃NO₆ (M+H⁺) 516.0, found 516.9.

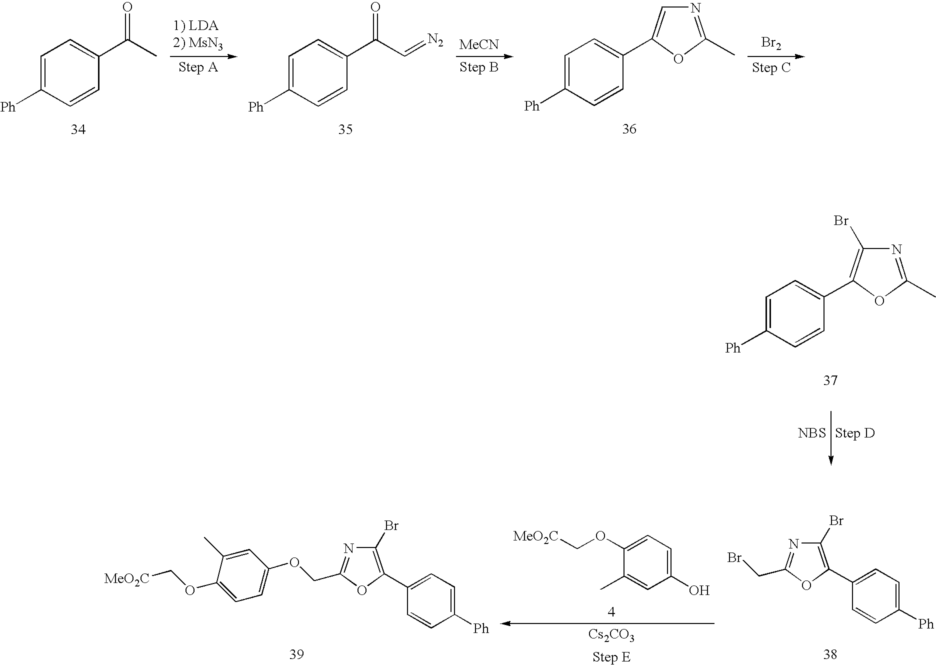

### Intermediate 39

### [4-(5-Biphenyl-4-yl-4-bromo-oxazol-2-ylmethoxy)-2-methyl-phenoxy]-acetic acid methyl ester

Step A: Following the procedure of Intermediate 33, step A, except substituting 4′-phenylacetophenone 34 for 4′-(trifluoromethoxy)acetophenone 28 in step A, 1-biphenyl-4-yl-2-diazo-ethanone 35 (7.20 g, 43%) was obtained as a yellow solid: ¹H-NMR (400 MHz, CDCl₃) δ=7.72 (d, J=8.4 Hz, 2H), 7.55 (d, J=8.4 Hz, 2H), 7.49 (d, J=7.2 Hz, 2H), 7.35-7.24 (m, 3H), 5.81 (s, 1H). MS calcd. for C₁₄H₁₁N₂O (M⁺) 223.0, found 195.0 (M+H⁺—N₂).

Step B: Following the procedure of Intermediate 33, step B, 5-biphenyl-4-yl-2-methyl-oxazole 36 (6.05 g, 80%) was obtained as a white solid: ¹H-NMR (400 MHz, CDCl₃) δ=7.70-7.61 (m, 6H), 7.48-7.36 (m, 3H), 7.25 (s, 1H), 2.58 (s, 3H). MS calcd. for C₁₆H₁₄NO (M+H⁺) 236.1, found 236.0.

Step C: Following the procedure of Intermediate 33, step C, 5-biphenyl-4-yl-4-bromo-2-methyl-oxazole 37 (1.45 g, 54%) was obtained as a light yellow solid: ¹H-NMR (400 MHz, CDCl₃) δ=7.80 (d, J=8.8 Hz, 2H), 7.51 (d, J=8.8 Hz, 2H), 7.46 (d, J=8.4 Hz, 2H), 7.30-7.17 (m, 3H), 2.36 (s, 3H). MS calcd. for C₁₆H₁₃BrNO (M+H⁺) 314.0, found 313.9.

Step D: Following the procedure of Intermediate 33, step D, 5-biphenyl-4-yl-4-bromo-2-bromomethyl-oxazole 38 (1.36 g, 79%) was obtained as a light yellow solid:

¹H-NMR (400 MHz, CDCl₃) δ=7.95 (d, J=8.4 Hz, 2H), 7.64 (d, J=8.8 Hz, 2H), 7.57 (d, J=7.2 Hz, 2H), 7.42-7.32(m, 3H), 4.43 (s, 3H). MS calcd. for C₁₆H₁₂Br₂NO (M+H⁺) 391.9, found 391.9.

Step E: Following the procedure of Intermediate 33, step E, [4-(5-Biphenyl-4-yl-4-bromo-oxazol-2-ylmethoxy)-2-methyl-phenoxy]-acetic acid methyl ester 39 (492 mg, 36%) was obtained as a light yellow solid: ¹H-NMR (400 MHz, CDCl₃) δ=8.21 (d, J=8.4 Hz, 2H), 7.89 (d, J=8.4 Hz, 2H), 7.83 (d, J=8.0 Hz, 2H), 7.68-7.56 (m, 3H), 7.08 (d, J=3.2 Hz, 1H), 7.00-6.85 (m, 2H), 5.30 (s, 2H), 4.80 (s, 2H), 3.99 (s, 3H), 2.48 (s, 3H). MS calcd. for C₂₆H₂₃BrNO₅ (M+H⁺) 508.1, found 508.0.

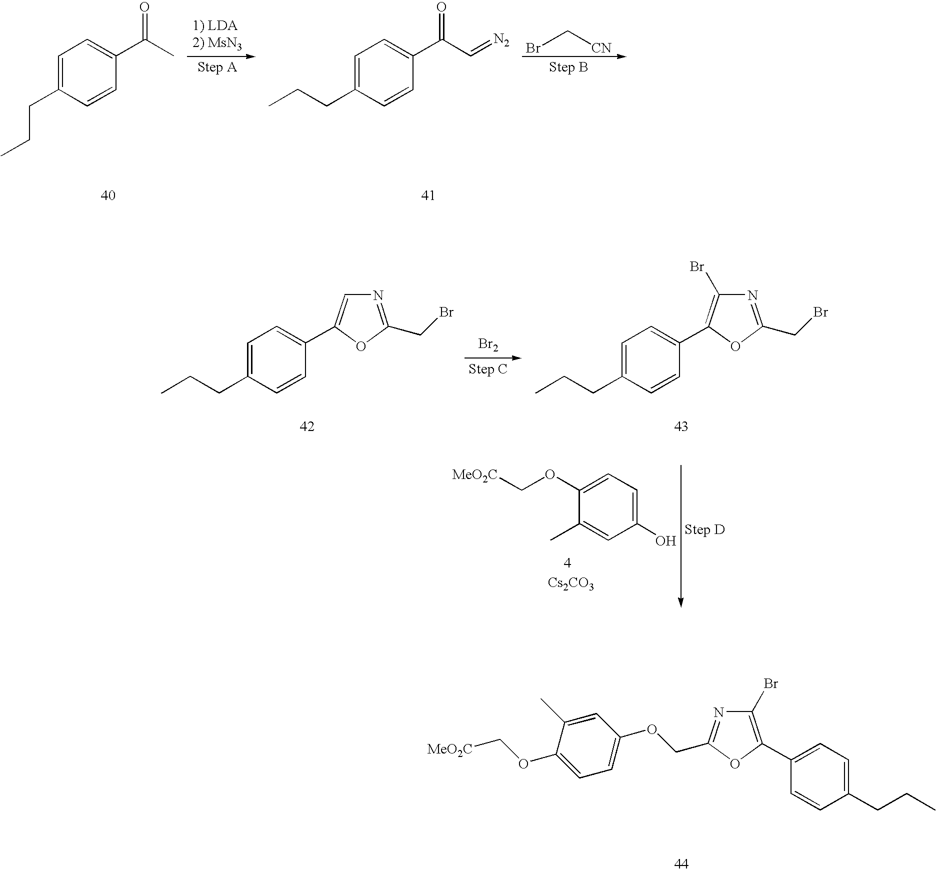

### Intermediate 44

### {4-[4-Bromo-5-(4-propyl-phenyl)-oxazol-2-ylmethoxy]-2-methyl-phenoxy}-acetic acid methyl ester

Step A: Following the procedure of Intermediate 33, step A, except substituting 4′-propylacetophenone 40 for 4′-(trifluoromethoxy)acetophenone 28 in step A, 2-diazo-4′-propylacetophenone 41 (16.2 g, 93%) was obtained as a yellow solid: ¹H-NMR (400 MHz, CDCl₃) δ=7.62 (d, J=8.4 Hz, 2H), 7.19 (d, J=8.4 Hz, 2H), 5.81 (s, 1H), 2.56 (t, J=7.8 Hz, 2H), 1.62-1.54 (m, 2H), 0.87 (t, J=7.2 Hz, 3H). MS calcd. for C₁₁H₁₃N₂O (M+H⁺) 189.0, found 161.1(M—N₂+H⁺).

Step B: Following the procedure of Intermediate 33, step B, except substituting bromoacetonitrile for acetonitrile. 2-Bromomethyl-5-(4-propyl-phenyl)-oxazole 42 (5.3 g, 72%) was obtained as a white solid: ¹H-NMR (400 MHz, CDCl₃) δ=7.34 (d, J=8.4 Hz, 2H), 7.04 (s, 11H), 7.02 (d, J=8.4 Hz, 2H), 4.44-4.30 (m, 2H), 2.38 (t, J=7.6 Hz, 2H), 1.47-1.38 (m, 2H), 0.72 (t, J=7.4 Hz, 3H). MS calcd. for C₁₃H₁₅BrNO (M+H⁺) 280.0, found 280.0.

Step C: Following the procedure of Intermediate 33, step C, 4-Bromo-2-bromomethyl-5-(4-propyl-phenyl)-oxazole 43 (2.7 g, 53%) was obtained as a light yellow solid: ¹H-NMR (400 MHz, CDCl₃) δ=7.85 (d, J=8.4 Hz, 2H), 7.28 (d, J=8.0 Hz, 2H), 4.63-4.48 (m, 2H), 2.63 (t, J=7.6 Hz, 2H), 1.71-1.62 (m, 2H), 0.96 (t, J=7.4 Hz, 3H). MS calcd. for C₁₃H₁₄Br₂NO (M+H⁺) 357.9, found 357.9.

Step D: Following the procedure of Intermediate 33, step E, {4-[4-Bromo-5-(4-propyl-phenyl)-oxazol-2-ylmethoxy]-2-methyl-phenoxy}-acetic acid methyl ester 44 (1.4 g, 100%) was obtained as a light yellow solid: ¹H-NMR (400 MHz, CD₃OD) δ=7.71 (d, J=8.4 Hz, 2H), 7.19 (d, J=8.4 Hz, 2H), 6.78 (d, J=2.8 Hz, 1H), 6.70-6.61 (m, 2H), 5.00 (s, 2H), 4.54 (s, 2H), 3.65 (s, 3H), 2.51 (t, J=7.6 Hz, 2H), 1.60-1.51 (m, 2H), 0.84 (t, J=7.2 Hz, 3H). MS calcd. for C₂₃H₂₅BrNO₅ (N+H⁺) 474.1, found 474.0.

### Intermediate 45

### 2-Isopropoxy-5-pyridineboronic acid

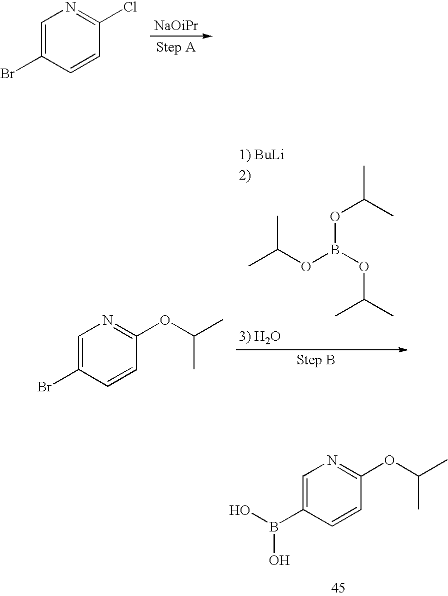

Step A: NaH (5.2 g, 130 mmol) was suspended in isopropanol (50 mL). The mixture was stirred for 30 min at 60° C. After the gas evolution ceased, 2-chloro-5-bromopyridine (10.0 g, 52 μmol) dissolved in isopropanol (100 mL) was added and the mixture was heated to reflux for 24 h. The solvent was removed in vacuo, and the remainder was taken up in H₂O and extracted with EtOAc. The organic layer was seperated and dried over MgSO₄, filtered and concentrated to afford 2-isopropoxy-5-bromo-pyridine (8.4 g, 39 mmol, 75%) as a light brown oil: ¹H-NMR (400 MHz, CDCl₃) δ=8.10 (d, J=2.5 Hz, 1H), 7.54 (dd, J=2.5 Hz, J=8.8 Hz, 1H), 6.52 (d, J=8.8 Hz, 1H), 5.17 (m, 1H), 1.26 (d, J=6.2 Hz, 6H). MS calcd. for C₈H₁₁BrNO (M+H⁺) 216.0, found 215.9.

Step B: 2-Isopropoxy-5-bromo-pyridine (0.65 g, 3 mmol) was dissolved in dry ether (10 mL) and cooled to −78° C. under argon. Butyl lithium (1.6 M in hexane, 2.81 mL, 4.5 mmol) was added dropwise and the mixture was stirred at −78° C. for 2 h. Then triisopropyl borate (1.72 mL, 7.5 mmol) was added quickly and the mixture was stirred for another 2 h at −78° C. The mixture was allowed to warm to rt, quenched with H₂O (20 mL) and stirred overnight at rt. The ether was removed in vacuo, the aqueous layer was adjusted to pH 10 (with 2 M NaOH) and washed with ether. Then the aqueous layer was adjusted to pH 3 (with 48% aq. HBr) and extracted with EtOAc three times. The organic layer was seperated and dried over MgSO₄, filtered and concentrated to afford 2-isopropoxy-5-pyridineboronic acid 45 (0.42 g, 2.3 mmol, 77%) as a colourless glass: MS calcd. for C₈H₁₃BNO₃ (M+H⁺) 182.1, found 182.1.

### Intermediate 46

### 2-Isopropoxy-5-pyrimidineboronic acid

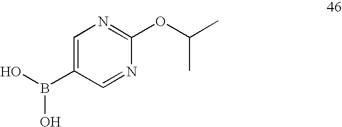

Following the procedure of Intermediate 45, except substituting 2-chloro-5-bromopyrimidine for 2-chloro-5-bromopyridine in Step A, the title compound was prepared as a white solid (0.15 g, 0.8 mmol, 27%): MS calcd. for C₇H 2BN₂O₃ (M+H⁺) 183.1, found 183.1.

### Intermediate 47

### 2-Morpholino-5-pyrimidineboronic acid

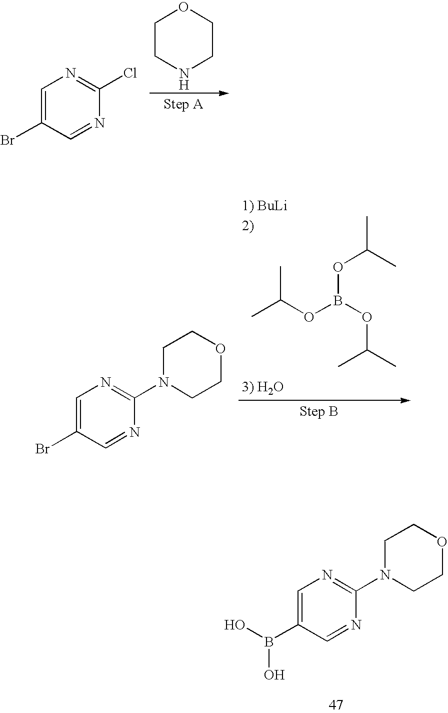

Step A: Morpholine (5.4 mL, 62.4 mmol) was dissolved in MeCN (250 mL). K₂CO₃ (8.6 g, 62.4 mmol) was added and the mixture was stirred at rt for 1 h. Then 2-chloro-5-bromo-pyrimidine (10.0 g, 52 mmol) was added and the mixture was heated to reflux for 5 h. The solvent was partially removed in vacuo and the remainder was taken up in H₂O and extracted with EtOAc. The organic layer was seperated and dried over MgSO₄, filtered and concentrated to afford 2-isopropoxy-5-bromo-pyrimidine (10.1 g, 41.1 mmol, 80%) as a light brown oil: ¹H-NMR (400 MHz, CDCl₃) δ=8.24 (s, 2H), 3.69 (m, 8H). MS calcd. for C₈H₁₁BrN₃O (M+H⁺) 244.0, found 243.9.

Step B: Following the procedure of Intermediate 45 Step B, except substituting 2-isopropoxy-5-bromo-pyrimidine for 2-isopropoxy-5-bromo-pyridine, the title compound was prepared as a white solid (0.38 g, 1.8 mmol, 60%): MS calcd. for C₈H₁₃BN₃O₃ (M+H⁺) 210.1, found 210.1.

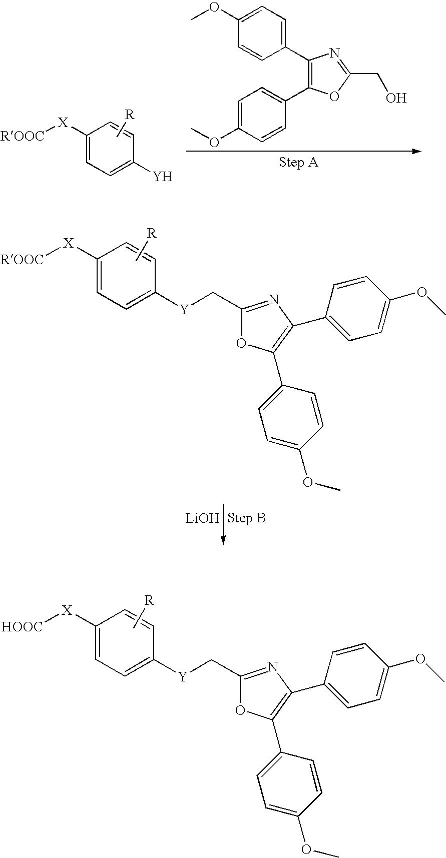

### Example A1

### {4-[4,5-Bis-(4-methoxy-phenyl)-oxazol-2-ylmethoxy]-2-methyl-phenoxy}-acetic acid

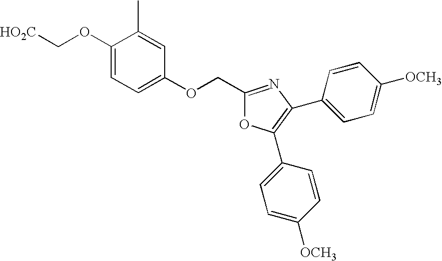

Step A: Intermediate 22 (25 mg, 0.08 mmol), intermediate 4 (18 mg, 0.09 mmol) and triphenylphosphine (30 mg, 0.11 mmol) were dissolved in dry DCM (1 mL) and cooled to 0° C. After the slow addition of diethyl azodicarboxylate (24 □L, 0.15 mmol) the solution was stirred at rt overnight. The solvent was removed to afford crude {4-[4,5-Bis-(4-methoxy-phenyl)-oxazol-2-ylmethoxy]-2-methyl-phenoxy}-acetic acid methyl ester which was used without further purification in step B.

Step B: The crude {4-[4,5-Bis-(4-methoxy-phenyl)-oxazol-2-ylmethoxy]-2-methyl-phenoxy}-acetic acid methyl ester was dissolved in THF (1 mL), a solution of 1 M LiOH in H₂O (0.2 mL) was added and the mixture was stirred overnight at rt. The mixture was acidified with 1 M HCl (0.25 mL), EtOAc (10 mL) was added and the organic layer washed with H₂O (3×5 mL). The organic layer was dried (MgSO₄), filtered, concentrated and purified on reverse phase HPLC(H₂O/MeCN gradient) to afford the title compound A1 (10.4 mg, 0.022 mmol, 27%) as a white solid: ¹H-NMR (400 MHz, CD₃OD) δ=7.40-7.37 (m, 4H), 6.81-6.87 (m, 5H), 6.74-6.66 (m, 2H), 5.04 (s, 2H), 4.52 (s, 2H), 3.73 (s, 3H), 3.72 (s, 3H), 2.16 (s, 3H). MS calcd. for C₂₇H₂₆NO₇ (M+H⁺) 476.16, found 476.10.

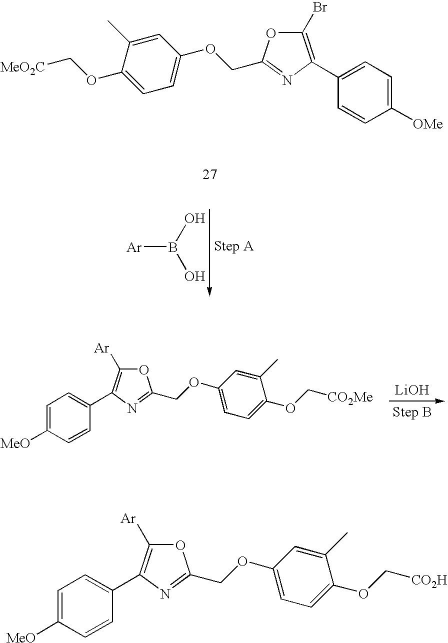

### Example B1

### {4-[5-Biphenyl-4-yl-4-(4-methoxy-phenyl)-oxazol-2-ylmethoxy]-2-methyl-phenoxy}-acetic acid

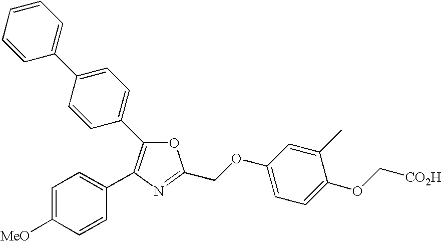

Step A: A mixture of {4-[5-bromo-4-(4-methoxy-phenyl)-oxazol-2-ylmethoxy]-2-methyl-phenoxy}-acetic acid methyl ester 27 (30.0 mg, 0.064 mmol), 4-biphenylboronic acid (25.7 mg, 0.13 mmol), tetrakis (triphenylphosphine) palladium (7.9 mg, 0.006 mmol), potassium carbonate (35.8 mg, 0.26 mmol), 1,4-dioxane (1 mL), EtOH (0.4 mL) and H₂O (0.2 mL) in a sealed vial was heated to 120° C. and stirred at this temperature overnight. The reaction mixture was cooled to room temperature and used in the next step without further purification. MS calcd. for C₃₃H₃₀NO₆ (M+H⁺) 536.2, found 536.2.

Step B: LiOH.H₂O (13.6 mg, 0.32 mmol) was added to the reaction mixture from step A. The mixture was stirred at room temperature for 2 h, and then filtered. The filtrate was purified on reverse phase HPLC(H₂O/MeCN gradient) to afford the title compound B1 (15.0 mg, 0.029 mmol, 45%) as a white solid: ¹H-NMR (400 MHz, CD₃OD) 8=7.63-7.57 (m, 6H), 7.49-7.47 (m, 2H), 7.38 (t, J=7.4 Hz, 2H), 7.28 (t, J=7.6 Hz, 1H), 6.93-6.91 (m, 2H), 6.87 (d, J=2.8 Hz, 1H), 6.79-6.69 (m, 2H), 5.11 (s, 2H), 4.54 (s, 2H), 3.75 (s, 3H), 2.17 (s, 3H). MS calcd. for C₃₂H₂₈NO₆ (M+H⁺) 522.2, found 522.2.

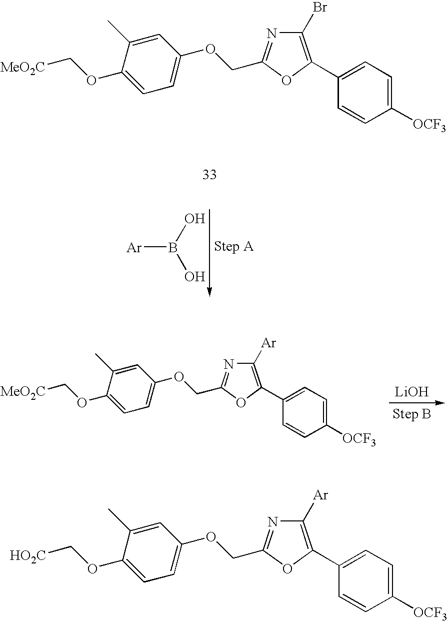

### Example C1

### {4-[4-(6-Isopropoxy-pyridin-3-yl)-5-(4-trifluoromethoxy-phenyl)-oxazol-2-ylmethoxy]-2-methyl-phenoxy}-acetic acid

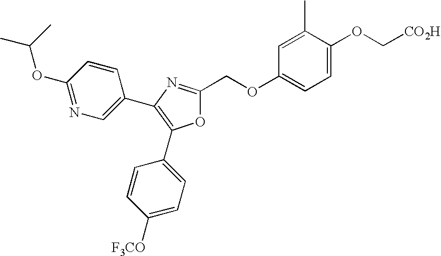

Step A: A mixture of {4-[4-bromo-5-(4-trifluoromethoxy-phenyl)-oxazol-2-ylmethoxy]-2-methyl-phenoxy}-acetic acid methyl ester 33 (150 mg, 0.29 mmol), 2-isopropoxy-5-pyridineboronic acid 45 (63.1 mg, 0.35 mmol), tetrakis triphenylphosphine) palladium (33.5 mg, 0.029 mmol) and potassium carbonate (1 M in H₂O, 1.2 mL, 1.2 mmol) was suspended in 1,4-dioxane (6.0 mL) and EtOH (3.0 mL). The mixture was heated to 120° C. in a sealed vial for 10 h, then cooled to room temperature and used in the next step without further purification. MS calcd. for C₂₉H₂₈F₃N₂O₇ (M+H⁺) 573.2, found 573.2.

Step B: LiOH.H₂O (61 mg, 1.45 mmol) was added to the reaction mixture from step A. The mixture was stirred at room temperature for 2 h, and then filtered. The filtrate was purified on reverse phase HPLC(H₂O/MeCN gradient) to afford the title compound Cl as a white solid (83.0 mg, 0.15 mmol, 52%): ¹H-NMR (400 MHz, CD₃OD) 6=8.36 (d, J=2.4 Hz, 1H), 7.90 (dd, J=8.8 Hz, J=2.4 Hz, 1H), 7.70 (d, J=9.2 Hz, 2H), 7.38 (d, J=8.0 Hz, 2H), 6.93 (d, J=3.2 Hz, 1H), 6.88-6.77 (m, 3H), 5.28 (m, 1H), 5.20 (s, 2H), 4.63 (s, 2H), 2.26 (s, 3H), 1.38 (d, J=6.0 Hz, 6H). MS calcd. for C₂₈H₂₆F₃N₂O₇ (M+H⁺) 559.2, found 558.9.

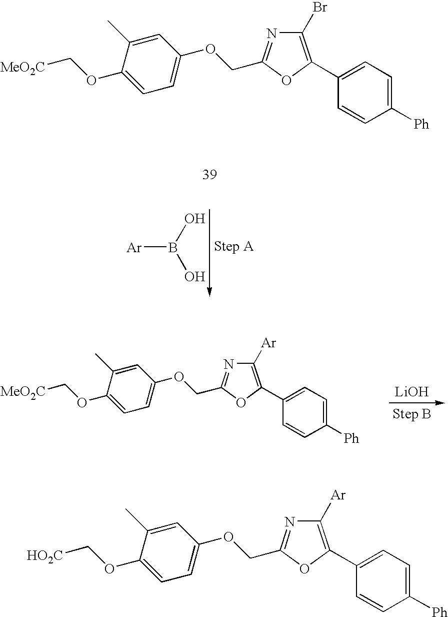

### Example D1

### {4-[5-Biphenyl-4-yl-4-(4-isopropoxy-phenyl)-oxazol-2-ylmethoxy]-2-methyl-phenoxy}-acetic acid

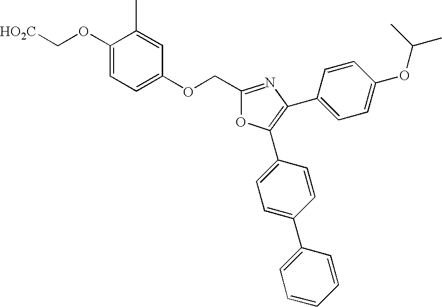

Following the procedure of example C1, except substituting intermediate 39 for intermediate 33, and substituting 4-isopropoxyphenylboronic acid for 2-isopropoxy-5-pyridineboronic acid in step A, the title compound D1 was prepared as a white solid (9.2 mg, 0.17 mmol, 43%): ¹H-NMR (400 MHz, CD₃OD) δ=7.59-7.55 (m, 6H), 7.44 (d, J=8.8 Hz, 2H), 7.35 (t, J=7.6 Hz, 2H), 7.26 (t, J=7.4 Hz, 1H), 6.88 (d, J=8.8 Hz, 2H), 6.85 (d, J=2.8 Hz, 2H), 6.79-6.68 (m, 2H), 5.08 (s, 2H), 4.56 (m, 1H), 4.52 (s, 2H), 2.17 (s, 3H), 1.26 (d, J=6.0 Hz, 6H). MS calcd. for C₃₄H₃₂NO₆ (M+H⁺) 550.2, found 550.2.

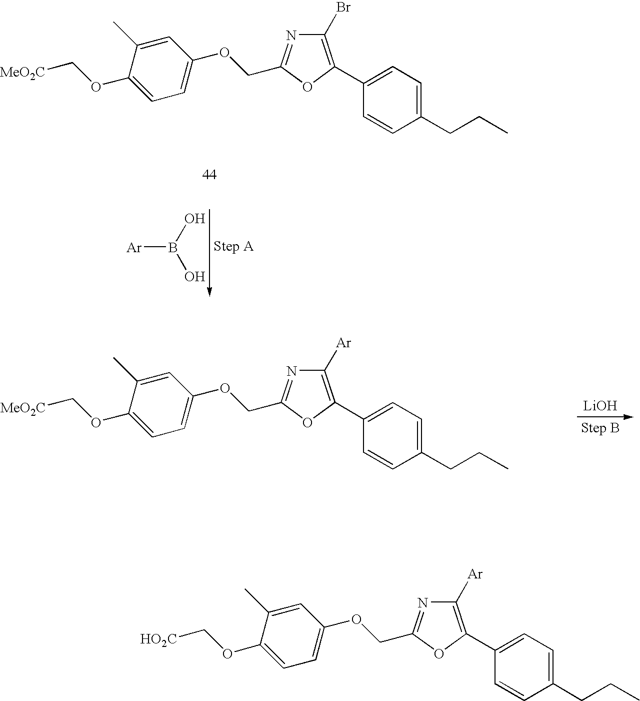

### Example E1

### {4-[4-(2-Methoxy-pyrimidin-5-yl)-5-(4-propyl-phenyl)-oxazol-2-ylmethoxy]-2-methyl-phenoxy}-acetic acid

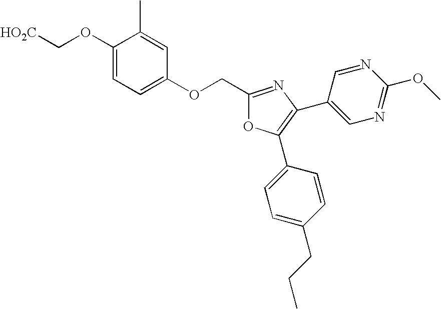

Following the procedure of Example D1, except substituting intermediate 44 for intermediate 33, and 2-methoxypyrimidine-5-boronic acid for 2-isopropoxy-5-pyridineboronic acid in step A, the title compound E1 was prepared as a white solid (8.6 mg, 0.018 mmol, 55%): ¹H-NMR (400 MHz, CD₃OD) δ=8.66 (s, 2H), 7.41 (d, J=8.0 Hz, 2H), 7.21 (d, J=8.0 Hz, 2H), 6.83 (d, J=2.8 Hz, 1H), 6.77-6.67 (m, 2H), 5.09 (s, 2H), 4.53 (s, 2H), 3.95 (s, 3H), 2.56 (t, J=7.6 Hz, 2H), 2.16 (s, 3H), 1.61-1.55 (m, 2H), 0.87 (t, J=7.4 Hz, 3H). MS calcd. for C₂₇H₂₈N₃O₆ (M+H⁺) 490.2, found 490.2.

By repeating the procedures described in the above examples, using appropriate starting materials, the following compounds of Formula I, as identified in Table 1, are obtained.

TABLE 1

| | | Physical Data |
|---|---|---|
| Compound | Compound | ¹H NMR 400 MHz (DMSO-d₆) |
| Number | Structure | and/or MS (m/z) |
| A2 | 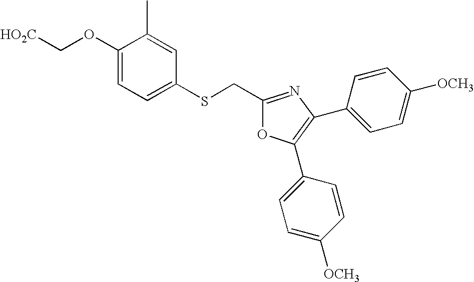 | ¹H-NMR (400 MHz, CD₃OD) δ =7.29(d, J=8.0 Hz, 2H), 7.23(d, J=8.4 Hz, 2H), 7.18(d, J=8.8 Hz, 1H), 7.13(s, 1H), 6.86-6.81(m, 4H), 6.67(d, J=8.4 Hz, 1H), 4.58 (s, 2H), 4.01(s, 2H), 3.73(s, 6H), 2.01(s, 3H). MS calcd. for C₂₇H₂₆NO₆S(M + H⁺) 492.14, found 492.10. |
| A3 | 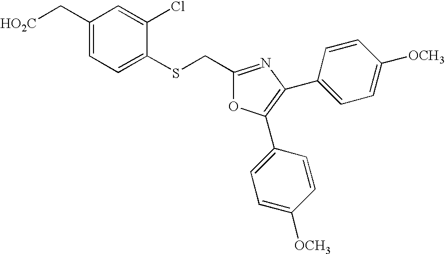 | ¹H-NMR (400 MHz, CD₃OD) δ =7.47(d, J=8.0 Hz, 1H), 7.33(d, J=1.6 Hz, 1H), 7.30(d, J=8.8 Hz, 2H), 7.23(d, J=8.8 Hz, 2H), 7.12 (dd, J=2.0 Hz, J=8.0 Hz, 1H), 6.79-6.85(m, 4H), 4.21(s, 2H), 3.72(s, 6H), 3.52(s, 2H). MS calcd. for C₂₆H₂₃C1NO₅S(M + H⁺) 496.09, found 496.00. |
| B2 | 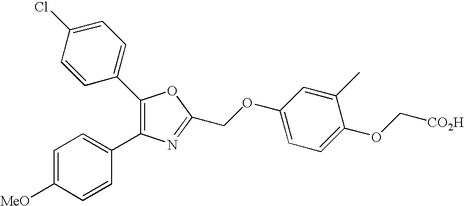 | ¹H-NMR (400 MHz, CD₃OD) δ =7.49(d, J=8.4 Hz, 2H), 7.44(d, J=8.8 Hz, 2H), 7.37(d, J=8.4 Hz, 2H), 6.92(d, J=8.8 Hz, 2H), 6.85(d, J=2.8 Hz, 1H), 6.79-6.69 (m, 2H), 5.09(s, 2H), 4.55(s, 2H), 3.75(s, 3H), 2.16(s, 3H). MS calcd. for C₂₆H₂₃C1NO₆ (M + H⁺) 480.1, found 480.1. |
| B3 | 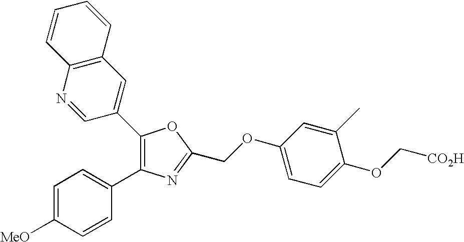 | ¹H-NMR (400 MHz, CD₃OD) δ =8.94(s, 1H), 8.61(s, 1H), 8.01- 7.95(m, 2H), 7.80-7.63(m, 2H), 7.53(d, J=8.8 Hz, 2H), 6.96(d, J=8.8 Hz, 1H), 6.89(d, J=3.2 Hz, 1H), 6.83-6.70(m, 2H), 5.17(s, 2H), 4.56(s, 2H), 3.76(s, 3H), 2.17(s, 3H). MS calcd. for C₂₉H₂₅N₂O₆ (M + H⁺) 497.2, found 497.2. |
| B4 | 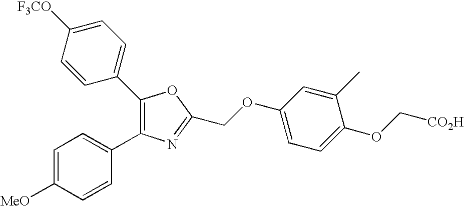 | ¹H-NMR (400 MHz, CD₃OD) δ =7.59(d, J=8.8 Hz, 2H), 7.43(d, J=9.2 Hz, 2H), 7.24(d, J=8.4 Hz, 2H), 6.92(d, J=8.8 Hz, 2H), 6.84(d, J=2.8 Hz, 1H), 6.79-6.68 (m, 2H), 5.08(s, 2H), 4.53(s, 2H), 3.75(s, 3H), 2.17(s, 3H). MS calcd. for C₂₇H₂₃F₃NO₇ (M + H⁺) 530.1, found 530.0. |
| B5 | 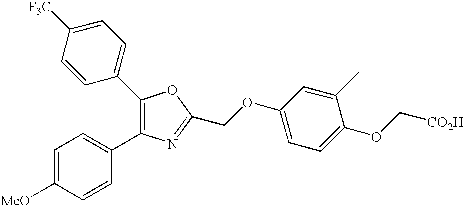 | ¹H-NMR (400 MHz, CD₃OD) δ =7.67-7.59(m, 4H), 7.43(d, J=8.8 Hz, 2H), 6.93(d, J=8.8 Hz, 2H), 6.85(d, J=2.8 Hz, 1H), 6.79-6.69 (m, 2H), 5.10(s, 2H), 4.53(s, 2H), 3.75(s, 3H), 2.17(s, 3H). MS calcd. for C₂₇H₂₃F₃NO₆ (M + H⁺) 514.1, found 514.1. |
| B6 | 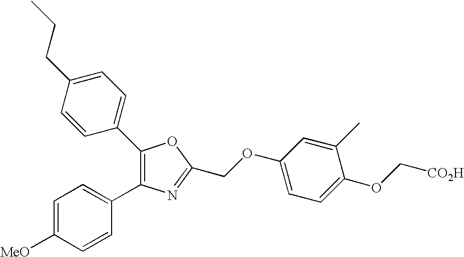 | ¹H-NMR (400 MHz, CD₃OD) δ =7.41-7.35(m, 4H), 7.11(d, J=8.4 Hz, 2H), 6.87(d, J=8.8 Hz, 2H), 6.82(d, J=2.4 Hz, 1H), 6.76-6.65 (m, 2H), 5.03(s, 2H), 4.51(s, 2H), 3.72(s, 3H), 2.50(t, J=7.6 Hz, 2H), 2.15(s, 3H), 1.59-1.50(m, 2H), 0.85(t, J=7.4 Hz, 3H). MS calcd. for C₂₉H₃₀NO₆ (M + H⁺) 488.2, found 488.2. |
| B7 | 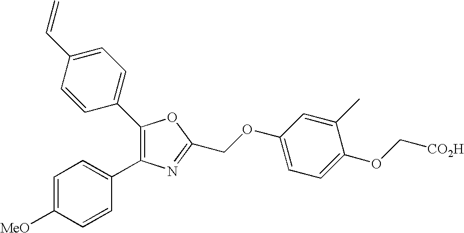 | ¹H-NMR (400 MHz, CD₃OD) δ =7.46-7.37(m, 6H), 6.90(d, J=8.8 Hz, 2H), 6.84(d, J=2.8 Hz, 2H), 6.78-6.62(m, 3H), 5.77(d, J=17.6 Hz, 1H), 5.21(d, J=10.8 Hz, 1H), 5.07(s, 2H), 4.54(s, 2H), 3.74(s, 3H), 2.16(s, 3H). MS calcd. for C₂₈H₂₆NO₆ (M + H⁺) 472.2, found 472.2. |
| B8 | 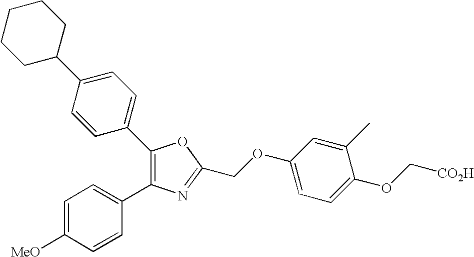 | ¹H-NMR (400 MHz, CD₃OD) δ =7.44-7.38(m, 4H), 7.17(d, J=8.0 Hz, 2H), 6.89(d, J=8.8 Hz, 2H), 6.84(d, J=2.8 Hz, 1H), 6.78-6.68 (m, 2H), 5.06(s, 2H), 4.54(s, 2H), 3.73(s, 3H), 2.50-2.42(m, 1H), 2.16(s, 3H), 1.77-1.65(m, 5H), 1.38-1.19(m, 5H). MS calcd. for C₃₂H₃₄NO₆ (M + H⁺) 528.2, found 528.2. |
| B9 | 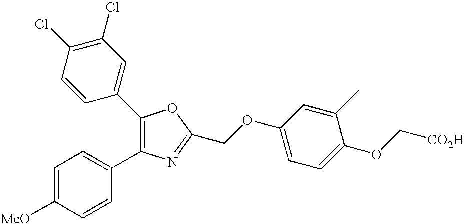 | ¹H-NMR (400 MHz, CD₃OD) δ =7.65(d, J=2.0 Hz, 1H), 7.52-7.40 (m, 4H), 6.95(d, J=8.4 Hz, 2H), 6.86(d, J=2.8 Hz, 1H), 6.80-6.69 (m, 2H), 5.11(s, 2H), 4.54(s, 2H), 3.76(s, 3H), 2.16(s, 3H). MS calcd. for C₂₆H₂₂C1₂NO₆ (M + H⁺) 514.1, found 514.1. |
| B10 | 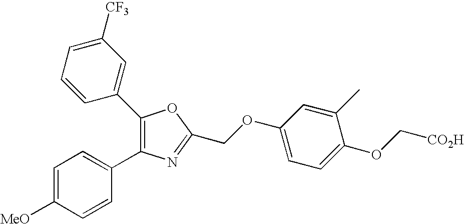 | MS calcd. for C₂₇H₂₃F₃NO₆(M + H⁺) 514.1, found 514.1. |
| B11 | 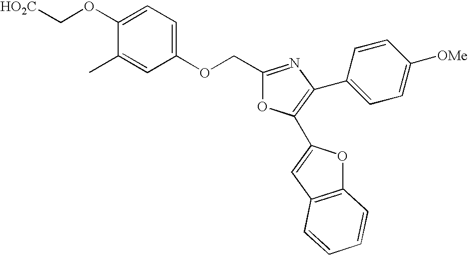 | ¹H-NMR (400 MHz, CD₃OD) δ =7.75-7.73(m, 2H), 7.60(d, J=7.6 Hz, 1H), 7.47(d, J=8.0 Hz, 1H), 7.30-7.19(m, 2H), 7.11(s, 1H), 6.99-6.96(m, 2H), 6.88(d, J=2.8 Hz, 1H), 6.82-6.70(m, 2H), 5.14 (s, 2H), 4.55(s, 2H), 3.78(s, 3H), 2.17(s, 3H). MS calcd. for C₂₈H₂₄NO₇ (M + H⁺) 486.2, found 486.1. |
| B12 | 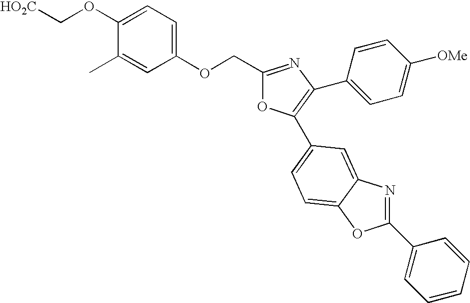 | ¹H-NMR (400 MHz, CD₃OD) δ =8.36-9.34(m, 2H), 8.00(s, 1H), 7.95(d, J=8.4 Hz, 1H), 7.77-7.66 (m, 6H), 7.13(d, J=8.8 Hz, 2H), 7.08(d, J=2.8 Hz, 2H), 7.00- 6.90(m, 2H), 5.33(s, 2H), 4.75(s, 2H), 3.93(s, 3H), 2.34(s, 3H). MS calcd. for C₃₃H₂₇N₂O₇ (M + H⁺) 563.2, found 563.2. |
| B13 | 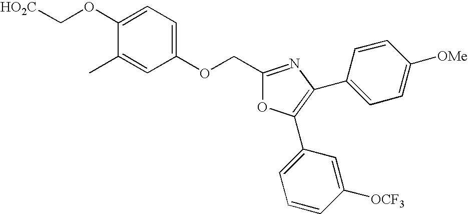 | ¹H-NMR (400 MHz, CD₃OD) δ =7.49-7.38(m, 4H), 7.33(s, 1H), 7.18(d, J=7.6 Hz, 1H), 6.91(d, J=8.8 Hz, 1H), 6.84(d, J=2.8 Hz, 1H), 6.78-6.67(m, 2H), 5.09(s, 2H), 4.53(s, 2H), 3.75(s, 3H), 2.16(s, 3H). MS calcd. for C₂₇H₂₃F₃NO₇ (M + H⁺) 530.1, found 530.1. |
| B14 | 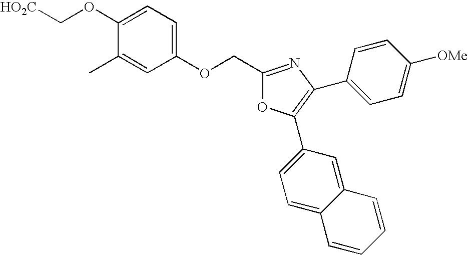 | ¹H-NMR (400 MHz, CD₃OD) δ =8.06(s, 1H), 7.84-7.79(m, 3H), 7.59-7.45(m, 5H), 6.93(d, J=8.4 Hz, 2H), 6.89(d, J=2.8 Hz, 1H), 6.83-6.71(m, 2H), 5.14(s, 2H), 4.56(s, 2H), 3.75(s, 3H), 2.17(s, 3H). MS calcd. for C₃₀H₂₆NO₆(M + H⁺) 496.2, found 496.2. |
| B15 | 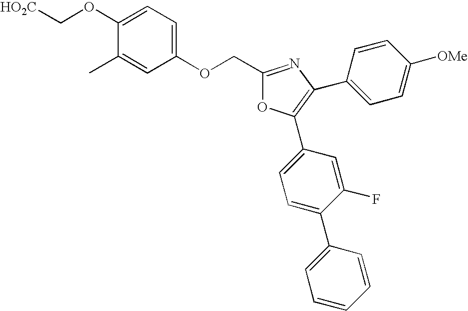 | ¹H-NMR (400 MHz, CD₃OD) δ =7.51-7.31(m, 9H), 6.96(d, J=8.8 Hz, 2H), 6.88(d, J=2.8 Hz, 1H), 6.82-6.70(m, 2H), 5.12(s, 2H), 4.56(s, 2H), 3.76(s, 3H), 2.17(s, 3H). MS calcd. for C₃₂H₂₇FNO₆(M + H⁺) 540.2, found 540.2. |
| B16 | 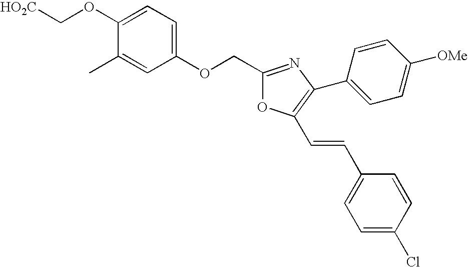 | ¹H-NMR (400 MHz, CD₃OD) δ 7.56(d, J=8.4 Hz, 2H), 7.45(d, J=8.4 Hz, 2H), 7.27(d, J=8.4 Hz, 2H), 7.13-7.04(m, 2H), 6.97(d, J=8.4 Hz, 2H), 6.84(d, J=2.4 Hz, 1H), 6.77-6.67(m, 2H), 5.05(s, 2H), 4.53(s, 2H), 3.76(s, 3H), 2.17(s, 3H). MS calcd. for C₂₈H₂₅C1NO₆ (M + H⁺) 506.1, found 506.1. |
| B17 | 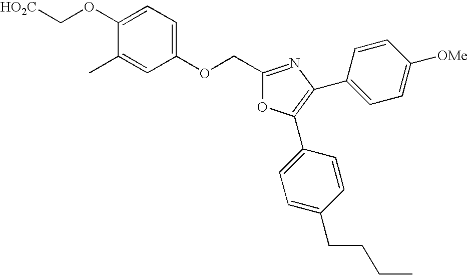 | ¹H-NMR (400 MHz, CD₃OD) δ =7.45-7.38(m, 4H), 7.15(d, J=8.4 Hz, 2H), 6.90(d, J=8.8 Hz, 2H), 6.86(d, J=2.8 Hz, 1H), 6.80-6.69 (m, 2H), 5.08(s, 2H), 4.55(s, 2H), 3.76(s, 3H), 2.56(t, J=7.8 Hz, 2H), 2.19(s, 3H), 1.58-1.22(m, 4H), 0.88(t, J=7.4 Hz, 3H). MS calcd. for C₃₀H₃₂NO₆ (M + H⁺) 502.2, found 502.2. |
| B18 | 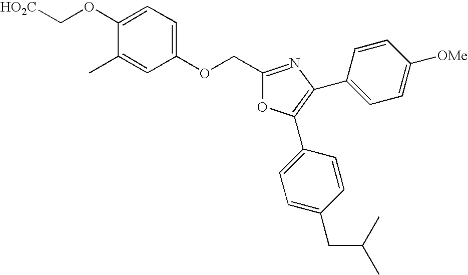 | ¹H-NMR (400 MHz, CD₃OD) δ =7.46-7.41(m, 4H), 7.14(d, J=8.0 Hz, 2H), 6.93(d, J=8.4 Hz, 2H), 6.88(d, J=2.8 Hz, 1H), 6.82-6.72 (m, 2H), 5.10(s, 2H), 4.57(s, 2H), 3.78(s, 3H), 2.46(d, J=7.2 Hz, 2H), 2.21(s, 3H), 1.90-1.79(m, 1H), 0.88(d, J=6.8 Hz, 3H). MS calcd. for C₃₀H₃₂NO₆ (M + H⁺) 502.2, found 502.2. |
| B19 | 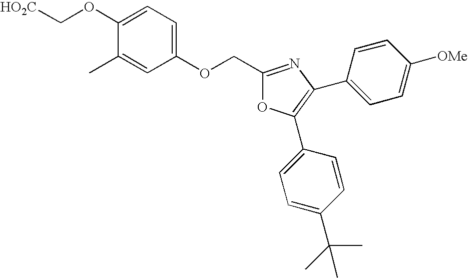 | ¹H-NMR (400 MHz, CD₃OD) δ =7.55-7.44(m, 6H), 6.99(d, J=8.4 Hz, 2H), 6.94(d, J=2.4 Hz, 1H), 6.88-6.78(m, 2H), 5.16(s, 2H), 4.62(s, 2H), 3.82(s, 3H), 2.24(s, 3H), 1.32(s, 9H). MS calcd. for C₃₀H₃₂NO₆ (M + H⁺) 502.2, found 502.2. |
| B20 | 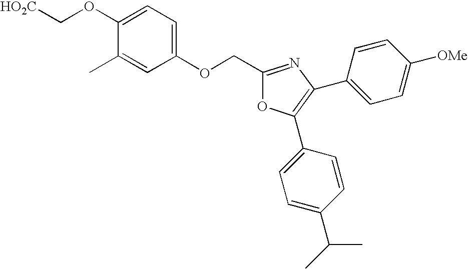 | ¹H-NMR (400 MHz, CD₃OD) δ =7.55-7.49(m, 4H), 7.31(d, J=8.0 Hz, 2H), 6.99(d, J=8.8 Hz, 2H), 6.94(d, J=2.4 Hz, 2H), 6.89-6.78 (m, 2H), 5.17(s, 2H), 4.63(s, 2H), 3.82(s, 3H), 2.96-2.89(m, 1H), 2.24(s, 3H), 1.26(d, J=7.2 Hz, 6H). MS calcd. for C₂₉H₃₀NO₆(M + H⁺) 488.2, found 488.1. |
| C2 | 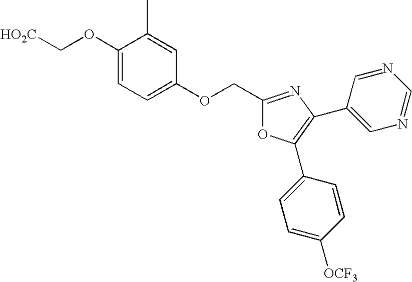 | ¹H-NMR (400 MHz, CD₃OD) δ =9.09(s, 1H), 8.92(s, 2H), 7.67(d, J=8.8 Hz, 2H), 7.37(d, J=8.4 Hz, 2H), 6.87(d, J=2.4 Hz, 1H), 6.81-6.70(m, 2H), 5.17(s, 2H), 4.55(s, 2H), 2.16(s, 3H). MS calcd. for C₂₄H₁₉F₃N₃O₆ (M + H⁺) 502.1, found 502.1. |
| C3 | 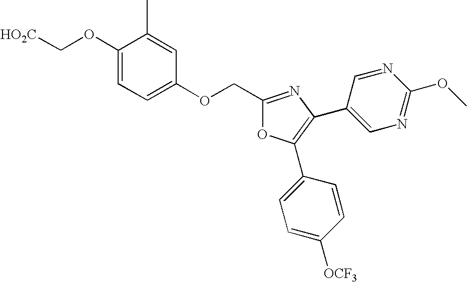 | ¹H-NMR (400 MHz, CD₃OD) δ =8.66(s, 2H), 7.62(d, J=8.8 Hz, 2H), 7.30(d, J=8.0 Hz, 2H), 6.83 (d, J=2.8 Hz, 1H), 6.77-6.66(m, 2H), 5.11(s, 2H), 4.53(s, 2H), 3.96(s, 3H), 2.15(s, 3H). MS calcd. for C₂₅H₂₁F₃N₃O₇ (M + H⁺) 532.1, found 532.1. |
| C4 | 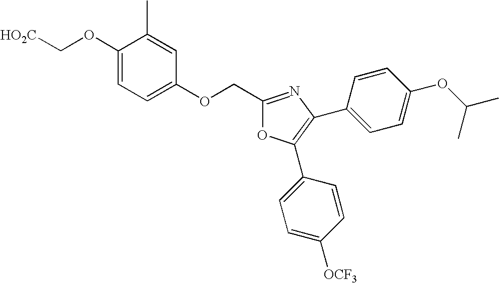 | ¹H-NMR (400 MHz, CD₃OD) δ =7.69(d, J=8.8 Hz, 2H), 7.50(d, J=8.8 Hz, 2H), 7.34(d, J=8.8 Hz, 2H), 6.98(d, J=8.8 Hz, 2H), 6.93 (d, J=3.2 Hz, 1H), 6.87-6.77(m, 2H), 5.18(s, 2H), 4.69(m, 1H), 4.59(s, 2H), 2.26(s, 3H), 1.35(d, J=6.0 Hz, 6H). MS calcd. for C₂₉H₂₇F₃NO₇ (M + H⁺) 558.2, found 558.2. |
| C5 | 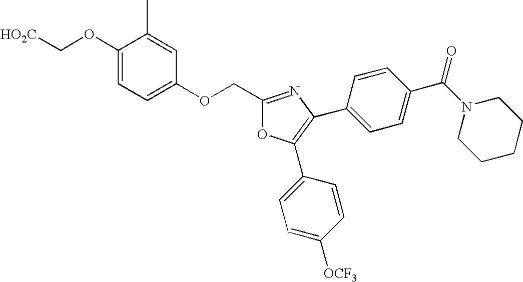 | ¹H-NMR (400 MHz, CD₃OD) δ =7.62-7.59(m, 4H), 7.38(d, J=8.4 Hz, 2H), 7.27(d, J=8.8 Hz, 2H), 6.84(d, J=2.8 Hz, 1H), 6.78-6.67 (m, 2H), 5.11(s, 2H), 4.53(s, 2H), 3.63(bs, 2H), 3.34(bs, 2H), 2.16 (s, 3H), 1.64-1.45(m, 6H). MS calcd. for C₃₂H₃₀F₃N₂O₇ (M + H⁺) 611.2, found 611.2. |
| C6 | 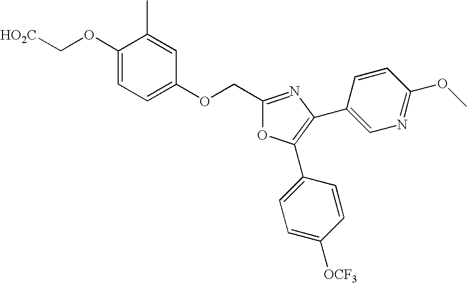 | ¹H-NMR (400 MHz, CD₃OD) δ =8.28(d, J=2.4 Hz, 1H), 7.79(dd, J=2.4 Hz, J=8.8 Hz, 1H), 7.60 (d, J=8.8 Hz, 2H), 7.30(d, J=8.4 Hz, 2H), 6.85(d, J=2.8 Hz, 1H), 6.81-6.68(m, 3H), 5.11(s, 2H), 4.54(s, 2H), 3.85(s, 3H), 2.16(s, 3H). MS calcd. for C₂₆H₂₂F₃N₂O₇ (M + H⁺) 531.1, found 531.1. |
| C7 | 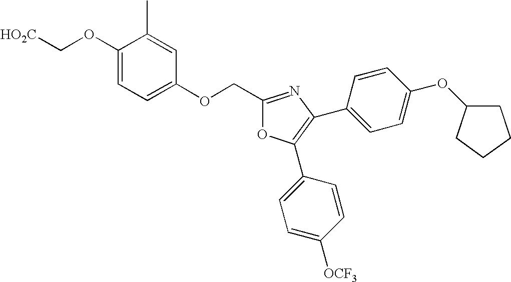 | ¹H-NMR (400 MHz, CD₃OD) δ =7.59(d, J=8.8 Hz, 2H), 7.40(d, J=8.8 Hz, 2H), 7.24(d, J=8.4 Hz, 2H), 6.87(d, J=8.4 Hz, 2H), 6.83 (d, J=2.8 Hz, 1H), 6.77-6.67(m, 2H), 5.08(s, 2H), 4.87-4.75(m, 1H), 4.48(s, 2H), 2.16(s, 3H), 1.96-1.58(m, 8H). MS calcd. for C₃₁H₂₉F₃NO₇ (M + H⁺) 584.2, found 584.1. |
| C8 | 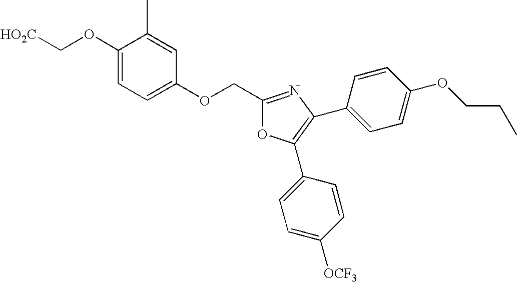 | ¹H-NMR (400 MHz, CD₃OD) δ =7.67(d, J=8.8 Hz, 2H), 7.49(d, J=8.8 Hz, 2H), 7.31(d, J=8.0 Hz, 2H), 6.99(d, J=8.8 Hz, 2H), 6.92 (d, J=2.8 Hz, 1H), 6.86-6.76(m, 2H), 5.16(s, 2H), 4.61(s, 2H), 3.97(t, J=6.4 Hz, 2H), 2.25(s, 3H), 1.84-1.78(m, 2H), 1.04(t, J=7.6 Hz, 3H). MS calcd. for C₂₉H₂₇F₃NO₇ (M + H⁺) 558.2, found 558.2. |
| C9 | 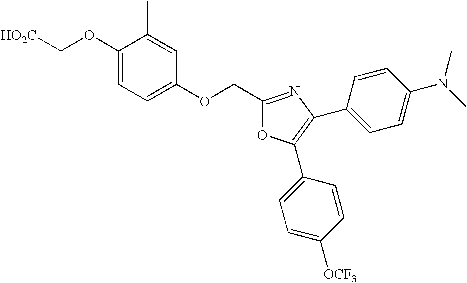 | MS calcd. for C₂₈H₂₆F₃N₂O₆(M + H⁺) 543.2, found 543.2. |
| C10 | 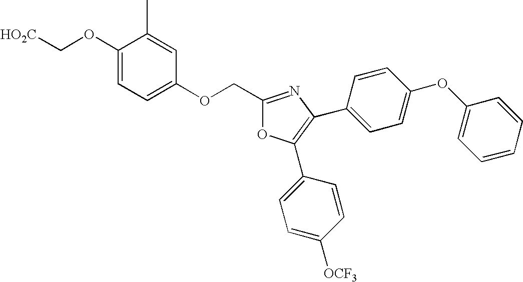 | ¹H-NMR (400 MHz, CD₃OD) δ =7.60(d, J=8.8 Hz, 2H), 7.49(d, J=8.8 Hz, 2H), 7.32-7.23(m, 4H), 7.07(t, J=7.4 Hz, 1H), 7.00-6.92 (m, 4H), 6.83(d, J=2.8 Hz, 1H), 6.78-6.67(m, 2H), 5.09(s, 2H), 4.53(s, 2H), 2.16(s, 3H). MS calcd. for C₃₂H₂₅F₃NO₇ (M + H⁺) 592.2, found 592.1. |
| C11 | 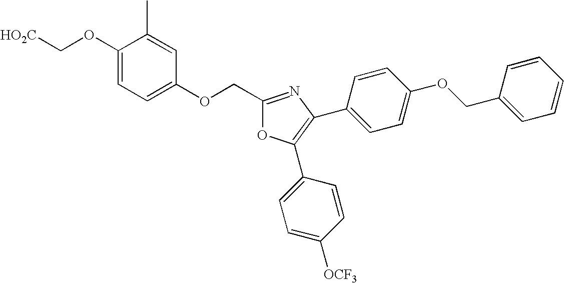 | ¹H-NMR (400 MHz, CD₃OD) δ =7.57(d, J=8.8 Hz, 2H), 7.42(d, J=8.8 Hz, 2H), 7.37-7.20(m, 7H), 6.98(d, J=8.8 Hz, 2H), 6.83(d, J=2.8 Hz, 1H), 6.77-6.67(m, 2H), 5.07(s, 2H), 5.04(s, 2H), 4.52(s, 2H), 2.16(s, 3H). MS calcd. for C₃₃H₂₇F₃NO₇ (M + H⁺) 606.2, found 606.1. |
| C12 | 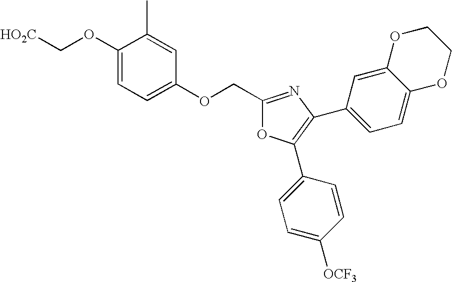 | ¹H-NMR (400 MHz, CD₃OD) δ =7.59(d, J=8.8 Hz, 2H), 7.24(d, J=8.0 Hz, 2H), 6.97-6.92(m, 2H), 6.83(d, J=2.8 Hz, 1H), 6.79-6.67 (m, 3H), 5.07(s, 2H), 4.53(s, 2H), 4.20-5.15(m, 4H), 2.16(s, 3H). MS calcd. for C₂₈H₂₃F₃NO₈(M + H⁺) 558.1, found 558.1. |
| C13 | 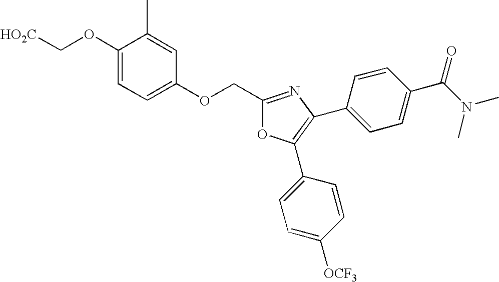 | ¹H-NMR (400 MHz, CD₃OD) δ =7.61-7.58(m, 4H), 7.41(d, J=8.8 Hz, 2H), 7.26(d, J=8.0 Hz, 2H), 6.83(d, J=3.2 Hz, 1H), 6.78-6.67 (m, 2H), 5.10(s, 2H), 4.52(s, 2H), 3.02(s, 3H), 2.95(s, 3H), 2.16(s, 3H). MS calcd. for C₂₉H₂₆F₃N₂O₇(M + H⁺) 571.2, found 571.1. |
| C14 | 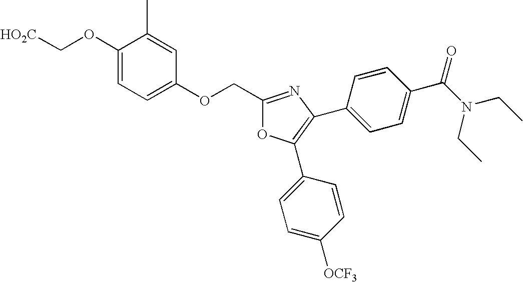 | ¹H-NMR (400 MHz, CD₃OD) δ =7.61-7.57(m, 4H), 7.36(d, J=8.0 Hz, 2H), 7.25(d, J=8.0 Hz, 2H), 6.83(d, J=2.8 Hz, 1H), 6.78-6.66 (m, 2H), 5.10(s, 2H), 4.52(s, 2H), 3.51-3.43(m, 4H), 2.16(s, 3H), 1.18-1.04(m, 6H). MS calcd. for C₃₁H₃₀F₃N₂O₇ (M + H⁺) 599.2, found 599.2. |
| C15 | 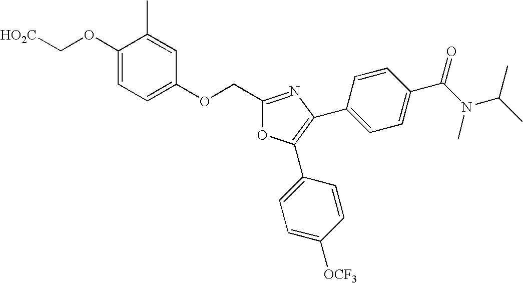 | ¹H-NMR (400 MHz, CD₃OD) δ =7.61-7.58(m, 4H), 7.40-7.31(m, 2H), 7.25(d, J=8.4 Hz, 2H), 6.83 (d, J=3.2 Hz, 1H), 6.78-6.67(m, 2H), 5.10(s, 2H), 4.52(s, 2H), 3.97-3.84(m, 1H), 2.86-2.78(m, 3H), 2.15(s, 3H), 1.19-1.09(m, 6H). MS calcd. for C₃₁H₃₀F₃N₂O₇(M + H⁺) 599.2, found 599.2. |
| C16 | 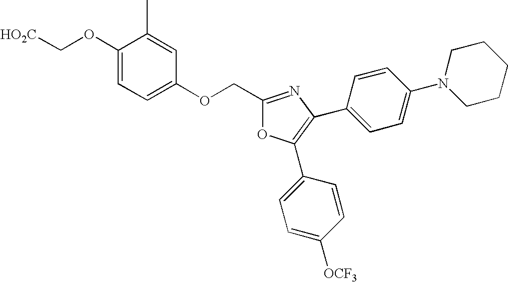 | ¹H-NMR (400 MHz, CD₃OD) δ =7.73-7.68(m, 4H), 7.50(d, J=8.8 Hz, 2H), 7.37(d, J=8.4 Hz, 2H), 6.93(d, J=2.8 Hz, 1H), 6.88-6.77 (m, 2H), 5.20(s, 2H), 4.63(s, 2H), 3.56-3.53(m, 4H), 2.26(s, 3H), 1.98-1.75(m, 6H), MS calcd. for C₃₁H₃₀F₃N₂O₆ (M + H⁺) 583.2, found 583.2. |
| C17 | 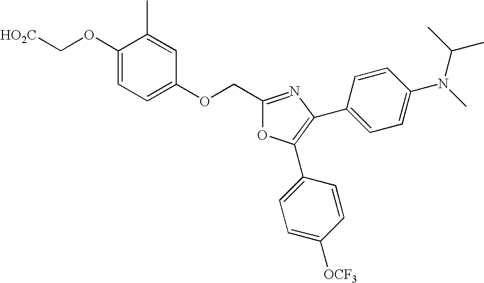 | ¹H-NMR (400 MHz, CD₃OD) δ =7.62-7.57(m, 4H), 7.26-7.21(m, 4H), 6.83(d, J=2.8 Hz, 1H), 6.77-6.66(m, 2H), 5.09(s, 2H), 4.53(s, 2H), 4.00-3.94(m, 1H), 3.01(s, 3H), 2.16(s, 3H), 1.20(s, 3H), 1.19(s, 3H). MS calcd. for C₃₀H₃₀ F₃N₂O₆ (M + H⁺) 571.2, found 571.2. |
| C18 | 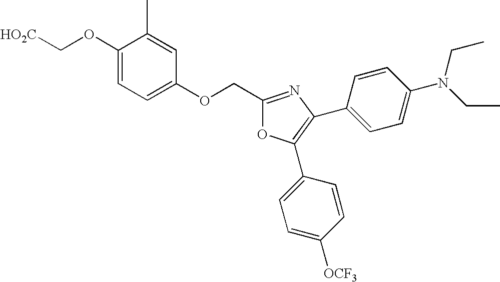 | MS calcd. for C₃₀H₃₀F₃N₂O₆(M + H⁺) 571.2, found 571.2. |
| C19 | 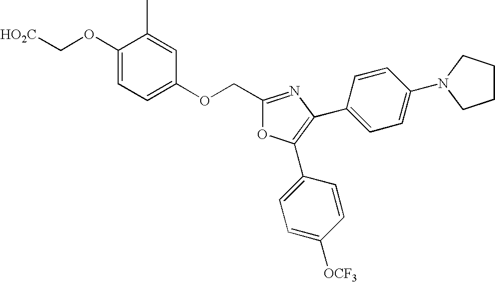 | ¹H-NMR (400 MHz, CD₃OD) δ =7.58(d, J=8.8 Hz, 2H), 7.31-7.17 (m, 4H), 6.82(d, J=2.8 Hz, 1H), 6.76-6.66(m, 2H), 5.04(s, 2H), 4.52(s, 2H), 3.24-3.07(m, 4H), 2.15(s, 3H), 1.96-1.93(m, 4H). MS calcd. for C₃₀H₂₈F₃N₂O₆(M + H⁺) 569.2, found 569.2. |
| C20 | 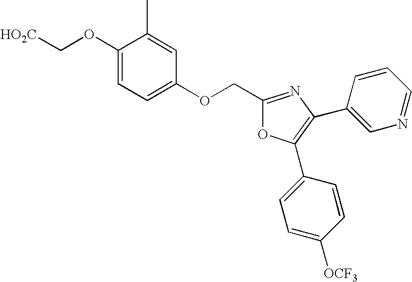 | ¹H-NMR (400 MHz, CD₃OD) δ =8.89(bs, 1H), 8.69(bs, 1H), 8.13 (d, J=7.6 Hz, 1H), 7.81(d, J=8.8 Hz, 2H), 7.61-7.52(m, 3H), 7.05(d, J=2.0 Hz, 1H), 6.99-6.88 (m, 2H), 5.33(s, 2H), 4.73(s, 2H), 2.33(s, 3H). MS calcd. for C₂₅H₂₀F₃N₂O₆ (M + H⁺) 501.1, found 501.1. |
| C21 | 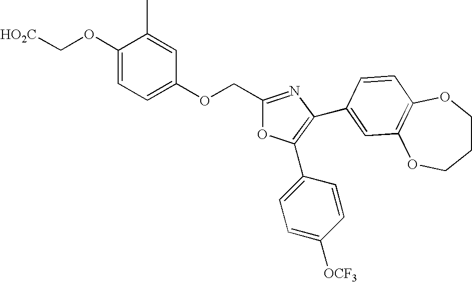 | ¹H-NMR (400 MHz, CD₃OD) δ =7.59-7.56(m, 2H), 7.24(d, J=8.0 Hz, 2H), 7.09-7.02(m, 2H), 6.90 (d, J=8.4 Hz, 1H), 6.83(d, J=2.8 Hz, 1H), 6.77-6.67(m, 2H), 5.07(s, 2H), 4.53(s, 2H), 4.15- 4.10(m, 4H), 2.16(s, 3H), 2.15- 2.05(m, 2H). MS calcd. for C₂₉H₂₅F₃NO₈ (M + H⁺) 572.2, found 572.1. |
| C22 | 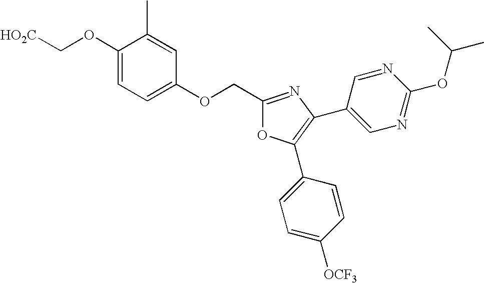 | ¹H-NMR (400 MHz, CDC1₃) δ =8.75(s, 2H), 7.96(d, J=8.7 Hz, 1H), 7.60(d, J=8.7 Hz, 1H), 7.30 (d, J=8.6 Hz, 1H), 7.25(d, J=8.6 Hz, 1H), 6.90-6.68(m, 3H), 5.32(m, 1H), 5.16(s, 2H), 4.64(s, 2H), 2.28(s, 3H), 1.42(d, J=6.2 Hz, 6H). MS calcd. for C₂₇H₂₅F₃N₃O₇ (M + H⁺) 602.1, found 602.1. |
| C23 | 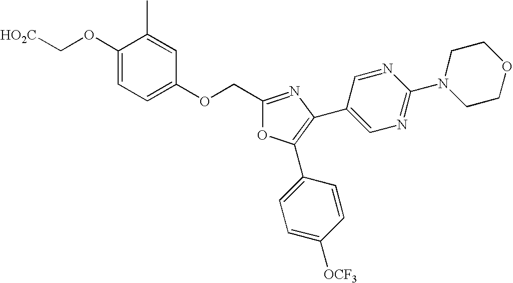 | ¹H-NMR (400 MHz, CDC1₃) δ =8.33(s, 2H), 7.37(d, J=8.3 Hz, 2H), 7.00(d, J=8.3 Hz, 2H), 6.64-6.43(m, 3H), 4.90(s, 2H), 4.38(s, 2H), 3.61(m, 4H), 3.55 (m, 4H), 2.02(s, 3H). MS calcd. for C₂₈H₂₆F₃N₄O₇ (M + H⁺) 587.2, found 587.2. |
| C24 | 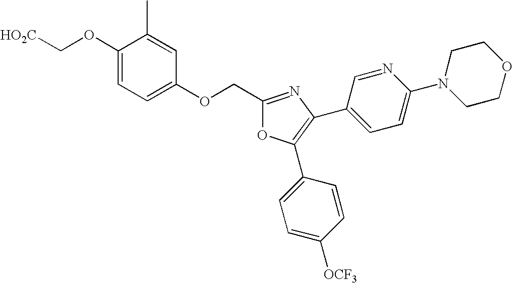 | ¹H-NMR (400 MHz, CDC1₃) δ =8.36(s, 1H), 7.37(dd, J=2.2 Hz, J=9.0 Hz, 1H), 7.58(d, J=8.5 Hz, 2H), 7.27(d, J=8.5 Hz, 2H), 6.91-6.68(m, 3H), 5.15(s, 2H), 4.64(s, 2H), 3.89(m, 4H), 3.72 (m, 4H), 2.26(s, 3H). MS calcd. for C₂₉H₂₇F₃N₃O₇ (M + H⁺) 586.2, found 586.3. |
| D2 | 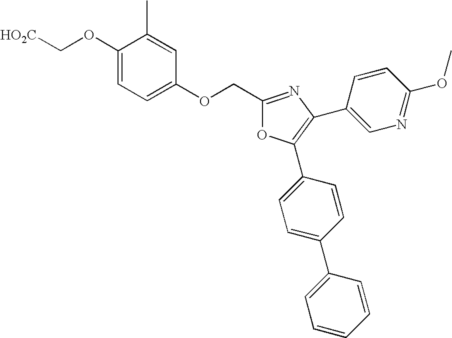 | ¹H-NMR (400 MHz, CD₃OD) δ =8.32(bs, 1H), 7.84(dd, J=8.8 Hz, J=2.0 Hz, 1H), 7.61-7.54(m, 6H), 7.36(t, J=7.4 Hz, 2H), 7.27 (t, J=7.2 Hz, 1H), 6.84-6.67(m, 4H), 5.10(s, 2H), 4.53(s, 2H), 3.87(s, 3H), 2.17(s, 3H). MS calcd. for C₃₁H₂₇N₂O₆ (M + H⁺) 523.2, found 523.2. |
| D3 | 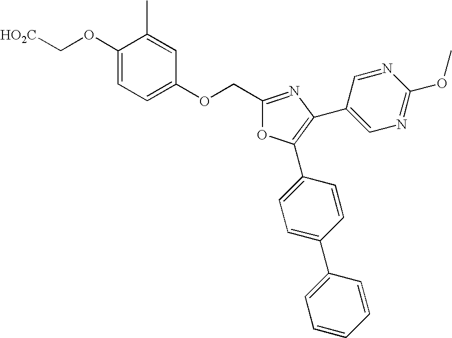 | ¹H-NMR (400 MHz, CD₃OD) δ =8.74(s, 2H), 7.75(d, J=8.4 Hz, 2H), 7.67(d, J=7.6 Hz, 2H), 7.61 (d, 8.4 Hz, 2H), 7.43(t, J=7.6 Hz, 2H), 7.33(t, J=7.2 Hz, 1H), 6.90(d, J=2.8 Hz, 1H), 6.83-6.71 (m, 2H), 5.20(s, 2H), 4.56(s, 2H), 3.91(s, 3H), 2.12(s, 3H). MS calcd. for C₃₀H₂₆N₃O₆ (M + H⁺) 524.2, found 524.2. |
| D4 | 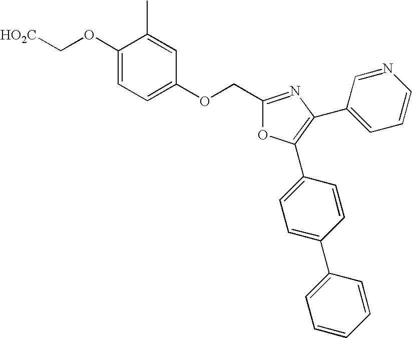 | ¹H-NMR (400 MHz, CD₃OD) δ =8.89(bs, 1H), 8.64(bs, 1H), 8.22 (d, J=8.0 Hz, 1H), 7.80-7.56(m, 7H), 7.49(t, J=7.4 Hz, 2H), 7.40 (t, J=7.0 Hz, 1H), 6.96(d, J=2.8 Hz, 1H), 6.91-6.79(m, 2H), 5.25 (s, 2H), 4.64(s, 2H), 2.24(s, 3H). MS calcd. for C₃₀H₂₅N₂O₆ (M + H⁺) 493.2, found 493.0. |
| D5 | 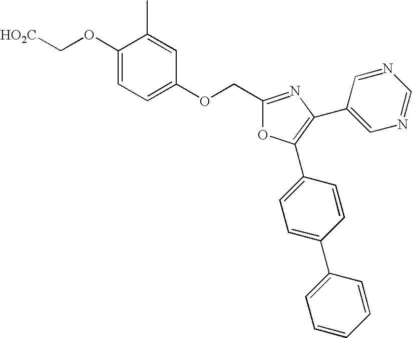 | ¹H-NMR (400 MHz, CD₃OD) δ =9.08(s, 1H), 8.96(s, 2H), 7.72- 7.31(m, 9H), 6.88(d, J=2.8 Hz, 1H), 6.80-6.70(m, 2H), 5.17(s, 2H), 4.56(s, 2H), 2.17(s, 3H). MS calcd. for C₃₆H₃₅N₂O₅ (M + H⁺) 494.2, found 494.1. |
| D6 | 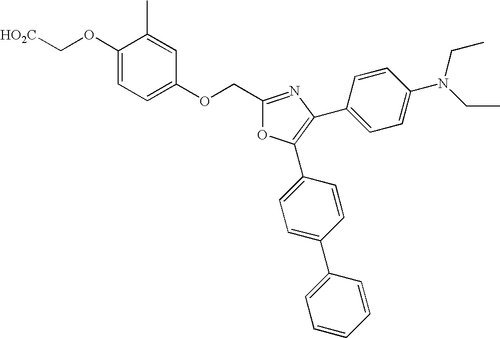 | ¹H-NMR (400 MHz, CD₃OD) δ =7.74(d, J=8.0 Hz, 2H), 7.63-7.56 (m, 6H), 7.38-7.26(m, 5H), 6.84 (d, J=2.8 Hz, 1H), 6.79-6.67(m, 2H), 5.11(s, 2H), 4.53(s, 2H), 3.56(q, J=7.2 Hz, 4H), 2.16(s, 3H), 1.09(t, J=7.2 Hz, 6H). MS calcd. for C₃₅H₃₅N₂O₅ (M + H⁺) 563.3, found 563.3. |
| D7 | 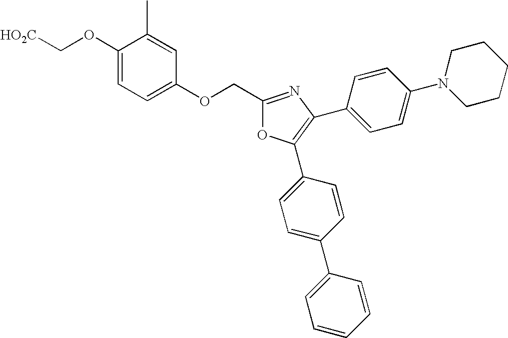 | ¹H-NMR (400 MHz, CD₃OD) δ =7.69(d, J=8.8 Hz, 2H), 7.62-7.55 (m, 6H), 7.42-7.26(m, 5H), 6.85 (d, J=2.8 Hz, 1H), 6.79-6.68(m, 2H), 5.11(s, 2H), 4.53(s, 2H), 3.47-3.45(s, 4H), 2.17(s, 3H), 1.90-1.66(s, 6H). MS calcd. for C₃₆H₃₅N₂O₅ (M + H⁺) 575.3, found 575.2. |
| D8 | 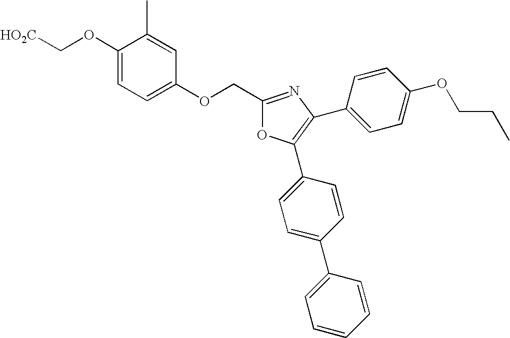 | ¹H-NMR (400 MHz, CD₃OD) δ =7.64-7.57(m, 6H), 7.48-7.29(m, 5H), 6.92(d, J=8.8 Hz, 2H), 6.87 (d, J=2.8 Hz, 1H), 6.81-6.70(m, 2H), 5.11(s, 2H), 4.56(s, 2H), 3.90(t, J=6.4 Hz, 2H), 2.17(s, 3H), 1.77-1.68(m, 2H), 0.97(t, J=7.4 Hz, 3H). MS calcd. for C₃₄H₃₂NO₆ (M + H⁺) 550.2, found 550.2. |
| D9 | 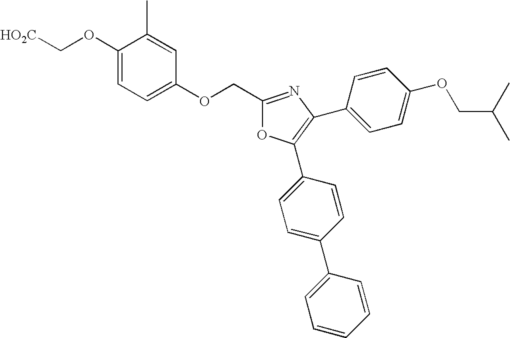 | ¹H-NMR (400 MHz, CD₃OD) δ =7.63-7.57(m, 6H), 7.49-7.27(m, 5H), 6.92(d, J=8.8 Hz, 2H), 6.87 (d, J=2.8 Hz, 1H), 6.81-6.70(m, 2H), 5.11(s, 2H), 4.56(s, 2H), 3.71(d, J=6.8 Hz, 2H), 2.17(s, 3H), 2.04-1.94(m, 1H), 0.97(d, J=6.8 Hz, 6H). MS calcd. for C₃₅H₃₄NO₆ (M + H⁺) 564.2, found 564.2. |
| D10 | 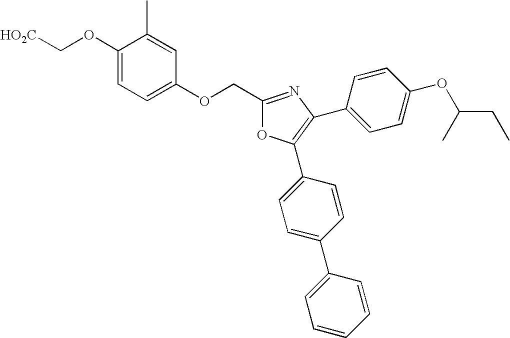 | ¹H-NMR (400 MHz, CD₃OD) δ =7.64-7.58(m, 6H), 7.48-7.27(m, 5H), 6.90(d, J=8.8 Hz, 2H), 6.87 (d, J=2.8 Hz, 1H), 6.81-6.70(m, 2H), 5.11(s, 2H), 4.54(s, 2H), 4.38-4.30(m, 1H), 2.17(s, 3H), 1.75-1.55(m, 2H), 1.22(d, J=6.0 HZ, 3H) 0.91(t, J=7.4 Hz, 3H). MS calcd. for C₃₅H₃₄NO₆ (M + H⁺) 564.2, found 564.2. |
| D11 | 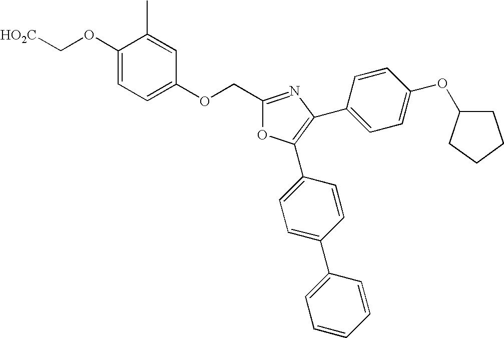 | ¹H-NMR (400 MHz, CD₃OD) δ =7.66-7.58(m, 6H), 7.47-7.27(m, 5H), 6.88-6.86(m, 3H), 6.81-6.70 (m, 2H), 5.11(s, 2H), 4.85-4.75 (m, 1H), 4.54(s, 2H), 2.17(s, 3H), 1.89-1.57(m, 8H). MS calcd. for C₃₆H₃₄NO₆ (M + H⁺) 576.2, found 576.2. |
| D12 | 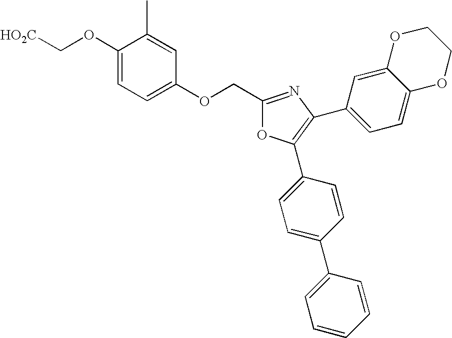 | ¹H-NMR (400 MHz, CD₃OD) δ =7.60-7.55(m, 6H), 7.37-7.24(m, 3H), 7.01-6.96(m, 2H), 6.84(d, J=2.8 Hz, 1H), 6.80-6.68(m, 4H), 5.07(s, 2H), 4.53(s, 2H), 4.20- 4.16(m, 4H), 2.17(s, 3H). MS calcd. for C₃₃H₂₈NO₇ (M + H⁺) 550.2, found 550.1. |
| D13 | 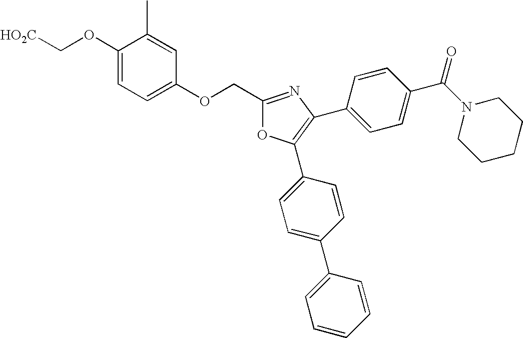 | ¹H-NMR (400 MHz, CD₃OD) δ =7.65-7.55(m, 8H), 7.37-7.25(m, 5H), 6.84(d, J=3.2 Hz, 1H), 6.79-6.67(m, 2H), 5.10(s, 2H), 4.52(s, 2H), 3.62(bs, 2H), 3.34 (bs, 2H), 2.16(s, 3H), 1.65-1.47 (m, 6H). MS calcd. for C₃₇H₃₅N₂O₆ (M + H⁺) 603.2, found 603.2. |
| D14 | 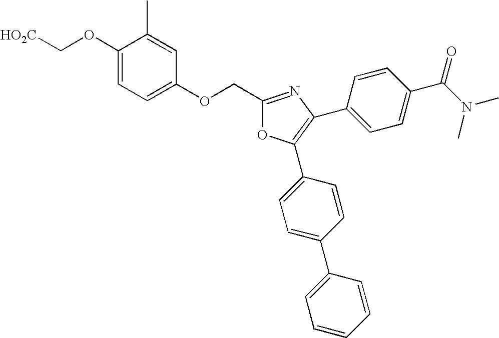 | ¹H-NMR (400 MHz, CD₃OD) δ =7.65(d, J=8.0 Hz, 2H), 7.60-7.54 (m, 5H), 7.40-7.33(m, 4H), 7.26 (t, J=7.4 Hz, 1H), 6.84(d, J=2.8 Hz, 1H), 6.79-6.67(m, 2H), 5.10 (s, 2H), 4.52(s, 2H), 3.02(s, 3H), 2.95(s, 3H), 2.16(s, 3H). MS calcd. for C₃₄H₃₁N₂O₆ (M + H⁺) 563.2, found 563.2. |
| D15 | 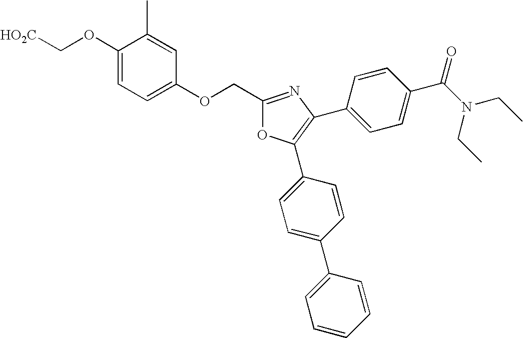 | ¹H-NMR (400 MHz, CD₃OD) δ =7.65(d, J=8.0 Hz, 2H), 7.62-7.53 (m, 5H), 7.39-7.33(m, 4H), 7.26 (t, J=7.4 Hz, 1H), 6.84(d, J=2.8 Hz, 1H), 6.79-6.66(m, 2H), 5.10 (s, 2H), 4.52(s, 2H), 3.52-3.15(m, 4H), 2.16(s, 3H), 1.21-1.00(m, 6H). MS calcd. for C₃₆H₃₅N₂O₆(M + H⁺) 591.2, found 591.2. |
| E2 | 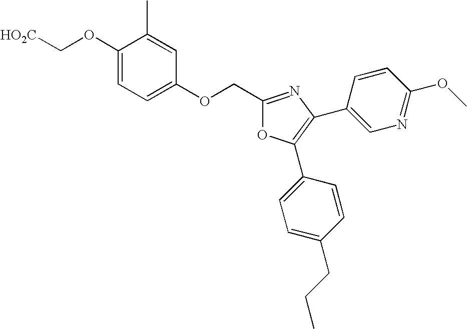 | ¹H-NMR (400 MHz, CD₃OD) δ =8.25(d, J=2.0 Hz, 1H), 7.78(dd, J=8.8 Hz, J=2.4 Hz, 1H), 7.38 (d, J=8.0 Hz, 2H), 7.17(d, J=8.4 Hz, 2H), 6.83(d, J=2.8 Hz, 1H), 6.77-6.66(m, 3H), 5.07(s, 2H), 4.52(m, 1H), 3.84(s, 3H), 2.52(t, J=7.6 Hz, 2H), 2.15(s, 3H), 1.59-1.53(m, 2H), 0.86(t, J=7.2 Hz, 3H). MS calcd. for C₂₈H₂₉N₂O₆ (M + H⁺) 489.2, found 489.1. |
| E3 | 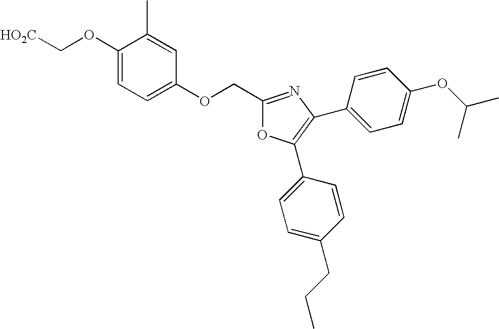 | ¹H-NMR (400 MHz, CD₃OD) δ =7.52-7.48(m, 4H), 7.26(d, J=8.0 Hz, 1H), 6.95-6.93(m, 3H), 6.89- 6.78(m, 2H), 5.16(s, 2H), 4.67- 4.61(m, 1H), 4.63(s, 2H), 2.59(t, J=7.6 Hz, 2H), 2.24(s, 3H), 1.68-1.62(m, 2H), 1.33(d, J=6.0 Hz, 6H), 0.95(t, J=7.4 Hz, 3H). MS calcd. for C₃₁H₃₄NO₆ (M + H⁺) 516.2, found 516.3. |
| E4 | 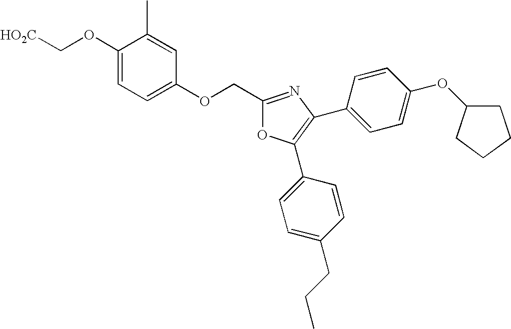 | ¹H-NMR (400 MHz, CD₃OD) δ =7.45(d, J=8.4 Hz, 4H), 7.19(d, J=8.0 Hz, 2H), 6.89-6.87(m, 3H), 6.83-6.73(m, 2H), 5.11(s, 2H), 4.83-4.80(m, 1H), 4.59(s, 2H), 2.57(t, J=7.6 Hz, 2H), 2.22(s, 3H), 1.97-1.58(m, 11H), 0.92(t, J=7.2 Hz, 3H). MS calcd. for C₃₃H₃₆NO₆ (M + H⁺) 542.3, found 542.3. |
| E5 | 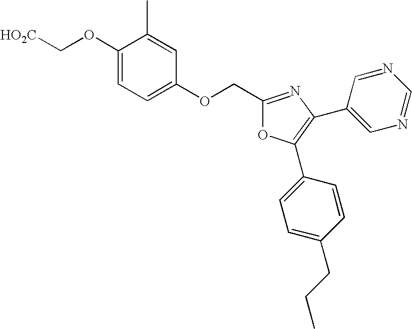 | ¹H-NMR (400 MHz, CD₃OD) δ =9.07(bs, 1H), 8.90(bs, 2H), 7.43 (d, J=8.0 Hz, 4H), 7.24(d, J=8.0 Hz, 2H), 6.84-6.70(m, 4H), 5.12(s, 2H), 4.52(s, 2H), 2.58- 2.54(m, 2H), 2.16(s, 3H), 1.63- 1.54(m, 2H), 0.87(t, J=7.4 Hz, 3H). MS calcd. for C₂₆H₂₆N₃O₅(M + H⁺) 460.2, found 460.1. |
| E6 | 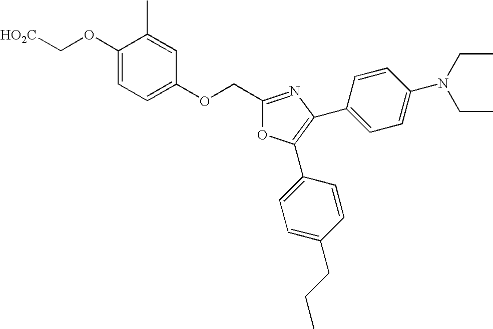 | MS calcd. for C₃₂H₃₇N₂O₅ (M + H⁺) 529.3, found 529.2. |

### Transcriptional Assay

Transfection assays are used to assess the ability of compounds of the invention to modulate the transcriptional activity of the PPARs. Briefly, expression vectors for chimeric proteins containing the DNA binding domain of yeast GAL4 fused to the ligand-binding domain (LBD) of either PPARδ PPARα or PPARγ are introduced via transient transfection into mammalian cells, together with a reporter plasmid where the luciferase gene is under the control of a GAL4 binding site. Upon exposure to a PPAR modulator, PPAR transcriptional activity varies, and this can be monitored by changes in luciferase levels. If transfected cells are exposed to a PPAR agonist, PPAR-dependent transcriptional activity increases and luciferase levels rise.

293T human embryonic kidney cells (8×10⁶) are seeded in a 175 cm² flask a day prior to the start of the experiment in 10% FBS, 1% Penicillin/Streptomycin/Fungizome, DMEM Media. The cells are harvested by washing with PBS (30 ml) and then dissociating using trypsin (0.05%; 3 ml). The trypsin is inactivated by the addition of assay media (DMEM, CA-dextran fetal bovine serum (5%). The cells are spun down and resuspended to 170,000 cells/ml. A Transfection mixture of GAL4-PPAR LBD expression plasmid (1 μg), UAS-luciferase reporter plasmid (1 μg), Fugene (3:1 ratio; 6 μL) and serum-free media (200 μL) was prepared and incubated for 15-40 minutes at room temperature. Transfection mixtures are added to the cells to give 0.16M cells/mL, and cells (50 μl/well) are then plated into 384 white, solid-bottom, TC-treated plates. The cells are further incubated at 37° C., 5.0% CO₂ for 5-7 hours. A 12-point series of dilutions (3 fold serial dilutions) are prepared for each test compound in DMSO with a starting compound concentration of 10 μM. Test compound (500 nl) is added to each well of cells in the assay plate and the cells are incubated at 37° C., 5.0% CO₂ for 18-24 hours. The cell lysis/luciferase assay buffer, Bright-Glom (25%; 25 μl; Promega), is added to each well. After a further incubation for 5 minutes at room temperature, the luciferase activity is measured.

Raw luminescence values are normalized by dividing them by the value of the DMSO control present on each plate. Normalized data is analyzed and dose-response curves are fitted using Prizm graph fitting program. EC50 is defined as the concentration at which the compound elicits a response that is half way between the maximum and minimum values. Relative efficacy (or percent efficacy) is calculated by comparison of the response elicited by the compound with the maximum value obtained for a reference PPAR modulator.

Compounds of Formula I, in free form or in pharmaceutically acceptable salt form, exhibit valuable pharmacological properties, for example, as indicated by the in vitro tests described in this application. Compounds of the invention preferably have an EC50 for PPARδ of less than 1 μM, more preferably less than 500 nm, more preferably less than 100 nM. Compounds of the invention are at least 100-fold selective for PPARδ over PPARγ.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A compound of Formula I: 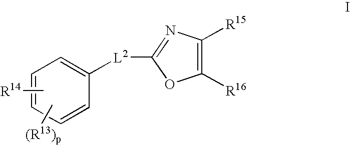
in which
p is an integer selected from 0 to 3;
L² is selected from —XOX—, —XS(O)₀₋₂X— and —XS(O)₀₋₂XO—; wherein X is independently selected from a bond and C₁₋₄alkylene; wherein any alkylene of L² can be optionally substituted by 1 to 3 radicals selected from halo, C₁₋₆alkyl, C₁₋₆alkoxy, halo-substituted-C₁₋₆alkyl and halo-substituted-C₁₋₆alkoxy;
R¹³ is selected from halo, C₁₋₆alkyl, C₁₋₆alkoxy, hydroxy-C₁₋₆alkyl, halo-substituted-C₁₋₆alkyl, halo-substituted-C₁₋₆alkoxy, C₆₋₁₀aryl, C₅₋₁₀heteraryl, C₃₋₁₂cycloalkyl and C₃₋₈heterocycloalkyl; wherein any aryl, heteroaryl, cycloalkyl and heterocycloalkyl of R¹³ is optionally substituted with 1 to 3 radicals independently selected from halo, nitro, cyano, C₁₋₆alkyl, C₁₋₆alkoxy, hydroxy-C₁₋₆alkyl, halo-substituted-C₁₋₆alkyl and halo-sub stituted-C₁₋₆ alkoxy;
R¹⁴ is selected from —XOXC(O)OR¹⁷ and —XC(O)OR¹⁷; wherein X is a bond or C₁₋₄alkylene; and R¹⁷ is selected from hydrogen and C₁₋₆alkyl;
R¹⁵ and R¹⁶ are independently selected from —R¹⁸ and —YR¹⁸; wherein Y is selected from C₁₋₆alkylene, C₂₋₆alkenylene, C₂₋₆alkynylene, —C(O)NR¹⁷— and —OX—; X is a bond or C₁₋₄alkylene; R¹⁷ is selected from hydrogen and C₁₋₆alkyl; and R¹⁸ is selected from C₃₋₁₂cycloalkyl, C₃₋₈heterocycloalkyl, C₆₋₁₀aryl and C₅₋₁₃heteroaryl; or R¹⁵ and R¹⁶ together with the atoms to which R¹⁵ and R¹⁶ are attached form fused bicyclic or tricyclic C₅₋₁₄heteroaryl;
wherein any aryl, heteroaryl, cycloalkyl and heterocycloalkyl of R¹⁸, or the combination of R¹⁵ and R¹⁶, is optionally substituted with 1 to 3 radicals independently selected from halo, nitro, cyano, C₁₋₆alkyl, C₁₋₆alkoxy, C₁₋₆alkylthio, hydroxy-C₁₋₆alkyl, halo-substituted-C₁₋₆alkyl, halo-substituted-C₁₋₆alkoxy, C₃₋₁₂cycloalkyl, C₃₋₈heterocycloalkyl, C₆₋₁₀aryl, C₅₋₁₃heteroaryl, —XS(O)₀₋₂R¹⁷, —XS(O)₀₋₂XR⁹, —XNR¹⁷R¹⁷, —X¹⁷S(₀₋₂R¹⁷, XNR¹⁷C(O)R¹⁷, XC(O)NR¹⁷R¹⁷, —XNR¹⁷C(O)R¹⁹, —XC(O)NR¹⁷R¹⁹, —XC(O)R¹⁹, —XNR¹⁷XR¹⁹ and —XOXR¹⁹; wherein any aryl, heteroaryl, cycloalkyl or heterocycloalkyl substituent is further optionally substituted with 1 to 3 radicals independently selected from halo, nitro, cyano, C₁₋₆alkyl, C₁₋₆alkoxy, C₁₋₆alkylthio, hydroxy-C₁₋₆alkyl, halo-substituted-C₁₋₆alkyl and halo-substituted-C₁₋₆alkoxy; wherein X is a bond or C₁₋₄alkylene; R¹⁷ is selected from hydrogen and C₁₋₆alkyl; and R¹⁹ is selected from C₃₋₁₂cycloalkyl, C₃₋₈heterocycloalkyl, C₆₋₁₀aryl and C₅₋₁₀heteroaryl; wherein any aryl, heteroaryl, cycloalkyl or heterocycloalkyl of R¹⁹ is optionally substituted with 1 to 3 radicals independently selected from halo, nitro, cyano, C₁₋₆alkyl, C₁₋₆alkoxy, halo-substituted-C₁₋₆alkyl and halo-substituted-C₁₋₆alkoxy; and the pharmaceutically acceptable salts, hydrates, solvates, isomers and prodrugs thereof.

2. The compound of claim 1 in which:
p is an integer selected from 0 to 3;
L² is selected from —XOX—, —XS(O)₀₋₂X— and —XS(O)₀₋₂XO—; wherein X is independently selected from a bond and C₁₋₄alkylene; wherein any alkylene of L² can be optionally substituted by 1 to 3 radicals selected from halo, C₁₋₆alkyl, C₁₋₆alkoxy, halo-substituted-C₁₋₆alkyl and halo-sub stituted-C₁₋₆ alkoxy;
R¹³ is C₁₋₆alkyl, C₁₋₆alkoxy and halogen; and
R¹⁴ is selected from —XOXC(O)OR¹⁷ and —XC(O)OR¹⁷; wherein X is a bond or C₁₋₄alkylene; and R¹⁷ is selected from hydrogen and C₁₋₆alkyl;
R¹⁵ and R¹⁶ are independently selected from —R¹⁸ and —YR⁸; wherein Y is selected from C₁₋₆alkylene, C₂₋₆alkenylene, —C(O)NR¹⁷— and —OX—; X is a bond or C₁₋₄alkylene; R¹⁷ is selected from hydrogen and C₁₋₆alkyl; and R¹⁸ is selected from C₆₋₁₀aryl, C₃₋₁₂cycloalkyl and C₅₋₁₃heteroaryl; or R¹⁵ and R¹⁶ together with the atoms to which R¹⁵ and R¹⁶ are attached form fused bicyclic or tricyclic C₅₋₁₄heteroaryl;
wherein any aryl, heteroaryl and cycloalkyl of R¹⁸, or the combination of R¹⁵ and R¹⁶, is optionally substituted with 1 to 3 radicals independently selected from halo, nitro, cyano, C₁₋₆alkyl, C₁₋₆alkoxy, C₁₋₆alkylthio, hydroxy-C₁₋₆alkyl, halo-substituted-C₁₋₆alkyl, halo-substituted-C₁₋₆alkoxy, C₃₋₁₂cycloalkyl, C₃₋₈heterocycloalkyl, C₆₋₁₀aryl optionally substituted with C₁₋₆alkoxy, C₅₋₁₃heteroaryl, —XS(O)₀₋₂R¹⁷, —XS(O)₂XR¹⁹, —XNR¹⁷R¹⁷, —XNR¹⁷S(₀₋₂R¹⁷, —XNR¹⁷C(O)R¹⁷, —XC(O)N¹⁷R¹⁷, —XNR¹⁷C(O)R⁹, —XC(O)NR¹⁷R¹⁹, —XC(O)R¹⁹, —XNR¹⁷XR¹⁹ and —XOXR¹⁹; wherein X is a bond or C₁₋₄alkylene; R¹⁷ is selected from hydrogen and C₁₋₆alkyl; and R¹⁹ is selected from C₆₋₁₀aryl, C₅₋₁₀heteroaryl, C₃₋₈heterocycloalkyl and C₃₋₁₂cycloalkyl; wherein any aryl, heteroaryl, cycloalkyl or heterocycloalkyl of R¹⁹ is optionally substituted with 1 to 3 radicals independently selected from halo, nitro, cyano, C₁₋₆alkyl, C₁₋₆alkoxy, halo-substituted-C₁₋₆alkyl and halo-substituted-C₁₋₆alkoxy.

3. The compound of claim 1 of Formula Ia: 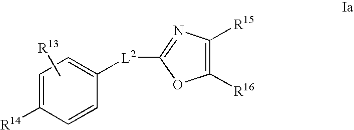
L² is selected from —S(O)₀₋₂(CH₂)₁₋₄O—, —O(CH₂)₁₄S(O)₀₋₂—, —CH₂S(O)₀₋₂—, —S(O)₀₋₂CH₂—, —S(O)₀₋₂—, —CH₂O— and —OCH₂—; I
R¹³ is selected from C₁₋₆alkyl, C₁₋₆alkoxy and halo;
R¹⁴ is selected from —OCH₂C(O)OH and —CH₂C(O)OH;
R¹⁵ and R¹⁶ are independently selected from —R^{1s} and —YR¹⁸; wherein Y is selected from C₁₋₆alkylene, C₂₋₆alkenylene, —C(O)NH— and —O(CH₂)₁₋₃—; and R¹⁸ is selected from phenyl, biphenyl, cyclohexyl, naphthyl, benzo[1,3]dioxol-5-yl, benzo[b]furanyl, pyridinyl, pyrimidinyl, dibenzo-furan-2-yl, furanyl, benzo[b]thiophene, thiophenyl, phenoxathiin-4-yl, benzoxazolyl, 3-oxo-3,4-dihydro-2H-benzo[1,4]oxazin-6-yl, 2-oxo-2,3-dihydro-benzooxazol-6-yl, 2,3-dihydro-benzo[1,4]dioxin-6-yl, benzoxazolyl, 3,4-dihydro-2H-benzo[b][1,4]dioxepin-7-yl and quinolinyl; or R¹⁵ and R¹⁶ together with the atoms to which R¹⁵ and R¹⁶ are attached form 4,5-dihydro-naphtho[1,2-d]thiazol-2-yl, 4H-chromeno[4,3-d]thiazol-2-yl, 5,6-dihydro-4H-3-thia-1-aza-benzo[e]azulen-2-yl, benzthiazolyl, benzoxazolyl and 1-oxa-3-aza-cyclopenta[a]naphthalen-2-yl;
wherein any aryl, heteroaryl, cycloalkyl and heterocycloalkyl of R¹⁵, R¹⁶ or the combination of R¹⁵ and R¹⁶, is optionally substituted with 1 to 3 radicals independently selected from halo, cyano, nitro, methyl, isopropyl, isopropyl-sulfanyl, isopropyloxy, hydroxy-methyl, methyl-sulfanyl, methoxy, ethoxy, pentafluoroethoxy, trifluoromethyl, trifluoromethoxy, trifluoromethyl-sulfonyl, morpholino, phenoxy, benzoxy, ethyl-sulfonyl, dimethylamino, methyl-sulfonyl-amino, ethyl-sulfonyl, propyl, vinyl, propyloxy, sec-butoxy, trifluoromethyl-sulfanyl, dimethyl-amino-carbonyl, diethyl-amino-carbonyl, methyl-carbonyl-amino, methyl-carbonyl, cyclopentyl-oxy, isopropyl-methylamino-carbonyl, cyclopropyl-amino-carbonyl, cyclohexyl, morpholino, piperidinyl, indolyl, pyrrolidinyl, pyrrolidinyl-carbonyl, 2,3-dihydro-benzofuran-5-yl piperidinyl-carbonyl, morpholino-carbonyl, isopropyl-methyl-amino, isopropyl-methyl-amino-carbonyl, diethyl-amino, and phenyl optionally substituted with methoxy.

4. The compound of claim 3 of Formula Ib: 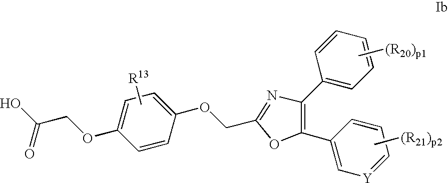
in which:
p1 and p2 are independently selected from 0, 1 and 2;
Y is selected from N and CH;
R¹³ is selected from C₁₋₆alkyl, C₁₋₆alkoxy and halo;
R²⁰ is selected from trifluoromethyl and trifluoromethoxy; and
R²¹ is selected from isopropyloxy and methoxy.

5. The compound of claim 4 that is {4-[4-(6-isopropoxy-pyridin-3-yl)-5-(4-trifluoromethoxy-phenyl)-oxazol-2-ylmethoxy]-2-methyl-phenoxy}-acetic acid.

6. A method for treating a disease or disorder in an animal in which modulation of PPARδ activity can prevent, inhibit or ameliorate the pathology and/or symptomology of the disease, which method comprises administering to the animal a therapeutically effective amount of a compound of claim 1.

7. The method of claim 6 in which the disease or disorder is selected from the treatment of prophylaxis, dyslipidemia, hyperlipidemia, hypercholesteremia, atherosclerosis, atherogenesis, hypertriglyceridemia, heart failure, myocardial infarction, vascular diseases, cardiovascular diseases, hypertension, obesity, cachexia, inflammation, arthritis, cancer, anorexia, anorexia nervosa, bulimia, Alzheimer's disease, skin disorders, respiratory diseases, ophthalmic disorders, irritable bowel diseases, ulcerative colitis, Crohn's disease, type-1 diabetes, type-2 diabetes and Syndrome X.

8. The method of claim 6 in which the disease or disorder is selected from HIV wasting syndrome, long term critical illness, decreased muscle mass and/or muscle strength, decreased lean body mass, maintenance of muscle strength and function in the elderly, diminished muscle endurance and muscle function, and frailty in the elderly.

9. The use of a compound according to any of claims 1 to 5 in the manufacture of a medicament for treating a disease in an animal in which PPARδ activity contributes to the pathology and/or symptomology of the disease.

10. A pharmaceutical composition comprising a therapeutically effective amount of a compound of any of claim 1 to 5 in combination with one or more pharmaceutically acceptable excipients.

11. A pharmaceutical combination, especially a pharmaceutical composition, comprising: 1) a compound of any of claims 1 to 5 or a pharmaceutical acceptable salt thereof; and 2) at least one active ingredient selected from:
a) anti-diabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; insulin sensitizer such as protein tyrosine phosphatase-1B (PTP-1B) inhibitors such as PTP-112; GSK3 (glycogen synthase kinase-3) inhibitors such as SB-517955, SB-4195052, SB-216763, N,N-57-05441 and N,N-57-05445; RXR ligands such as GW-0791 and AGN-194204; sodium-dependent glucose co-transporter inhibitors such as T-1095; glycogen phosphorylase A inhibitors such as BAY R3401; biguamides such as metformin; alpha-glucosidase inhibitors such as acarbose; GLP-1 (glucagon like peptide-1), GLP-1 analogs such as Exendin-4 and GLP-1 mimetics; dipeptidyl peptidase IV inhibitors such as DPP728, vildagliptin, MK-0431, saxagliptin, GSK23A; an AGE breaker; a thiazolidone derivative (glitazone) such as pioglitazone, rosiglitazone, or (R)-1-[4-[5-methyl-2-(4-trifluoromethyl-phenyl)-oxazol-4-ylmethoxy]-benzenesulfonyl}-2,3-dihydro-1H-indole-2-carboxylic acid, a non-glitazone type PPARγ agonist e.g. GI-262570;
b) hypolipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin;
c) an anti-obesity agent or appetite regulating agent such as phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine or cannabinoid receptor antagonists;
d) anti-hypertensive agents, e.g., loop diuretics such as ethacrynic acid, furosemide and torsemide; diuretics such as thiazide derivatives, chlorithiazide, hydrochlorothiazide, amiloride; angiotensin converting enzyme (ACE) inhibitors such as benazepril, captopril, enalapril, fosinopril, lisinopril, moexipril, perinodopril, quinapril, ramipril and trandolapril; inhibitors of the Na—K-ATPase membrane pump such as digoxin; neutralendopeptidase (NEP) inhibitors e.g. thiorphan, terteo-thiorphan, SQ29072; ECE inhibitors e.g. SLV306; ACE/NEP inhibitors such as omapatrilat, sampatrilat and fasidotril; angiotensin II antagonists such as candesartan, eprosartan, irbesartan, losartan, telmisartan and valsartan, in particular valsartan; renin inhibitors such as aliskiren, terlakiren, ditekiren, RO 66-1132, RO -66-1168; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors;
e) a HDL increasing compound;
f) a cholesterol absorption modulator such as Zetia® and KT6-971;
g) Apo-A1 analogues and mimetics;
h) thrombin inhibitors such as Ximelagatran;
i) aldosterone inhibitors such as anastrazole, fadrazole, eplerenone;
j) Inhibitors of platelet aggregation such as aspirin, clopidogrel bisulfate;
k) estrogen, testosterone, a selective estrogen receptor modulator, a selective androgen receptor modulator;
l) a chemotherapeutic agent such as aromatase inhibitors e.g. femara, anti-estrogens, topoisomerase I inhibitors, topoisomerase II inhibitors, microtubule active agents, alkylating agents, antineoplastic antimetabolites, platin compounds, compounds decreasing the protein kinase activity such as a PDGF receptor tyrosine kinase inhibitor preferably Imatinib or 4-Methyl-N-[3-(4-methyl-imidazol-1-yl)-5-trifluoromethyl-phenyl]-3-(4-pyridin-3-yl-pyrimidin-2-ylamino)-benzamide; and
m) an agent interacting with a 5-HT₃ receptor and/or an agent interacting with 5-HT₄ receptor such as tegaserod, tegaserod hydrogen maleate, cisapride, cilansetron;
or, in each case a pharmaceutically acceptable salt thereof; and optionally a pharmaceutically acceptable carrier.

12. A pharmaceutical composition according to claim 10 or a combination according to claim 11, for the treatment or prevention of dyslipidemia, hyperlipidemia, hypercholesteremia, atherosclerosis, hypertriglyceridemia, heart failure, myocardial infarction, vascular diseases, cardiovascular diseases, hypertension, obesity, inflammation, arthritis, cancer, Alzheimer's disease, skin disorders, respiratory diseases, ophthalmic disorders, inflammatory bowel diseases, 13Ds (irritable bowel disease), ulcerative colitis, Crohn's disease, conditions in which impaired glucose tolerance, hyperglycemia and insulin resistance are implicated, such as type-1 and type-2 diabetes, Impaired Glucose Metabolism (IGM), Impaired Glucose Tolerance (IGT), Impaired Fasting Glucose (IFG), and Syndrome-X.

13. A compound according to any of claims 1 to 5, or a pharmaceutical composition according to claim 10 or a combination according to claim 11, for use as a medicament.

14. Use of a compound according to any of claims 1 to 5, or a pharmaceutical composition according to claim 10 or a combination according to claim 11, for the manufacture of a medicament for the treatment or prevention of dyslipidemia, hyperlipidemia, hypercholesteremia, atherosclerosis, hypertriglyceridemia, heart failure, myocardial infarction, vascular diseases, cardiovascular diseases, hypertension, obesity, inflammation, arthritis, cancer, Alzheimer's disease, skin disorders, respiratory diseases, ophthalmic disorders, inflammatory bowel diseases, IBDs (irritable bowel disease), ulcerative colitis, Crohn's disease, conditions in which impaired glucose tolerance, hyperglycemia and insulin resistance are implicated, such as type-1 and type-2 diabetes, Impaired Glucose Metabolism (IGM), Impaired Glucose Tolerance (IGT), Impaired Fasting Glucose (IFG), and Syndrome-X.
